(19) United States
(12) Patent Application Publication
(10) Pub. No.: US 2006/0240451 A1
(43) Pub. Date: Oct. 26, 2006

(54) Compositions and methods employing 5' phosphate-dependent nucleic acid exonucleases

(75) Inventors: Jerome Jendrisak, Madison, WI (US); Judith Meis, Fitchburg, WI (US); Ronald Meis, Fitchburg, WI (US); Agnes Radek, Madison, WI (US); Gary Dahl, Madison, WI (US)

Correspondence Address: Medlen & Carroll, LLP;Suite 350, 101 Howard Street, San Francisco, CA, 94105, US

(73) Assignee: Epicentre Technologies, Madison, WI (US)

(21) Appl. No.: 11/352,172

(22) Filed: Feb. 9, 2006

Related U.S. Application Data
(60) Provisional application No. 60/651,409, filed on Feb. 9, 2005.
(60) Provisional application No. 60/685,367, filed on May 27, 2005.

(51) Int. Cl. C12Q 1/68; C07H 21/02; C12P 19/34

(52) U.S. Cl. 435/6; 435/91.2; 536/23.1

(57) ABSTRACT

The present invention relates to compositions and methods employing 5′-phosphate-dependent nucleic acid exonucleases. In particular, the present invention provides kits and methods employing 5′-phosphate-dependent nucleic acid exonucleases for selective enrichment, isolation and amplification of a particular set of desired nucleic acid molecules from samples that also contain undesired nucleic acid molecules for a variety of uses. In preferred embodiments, the desired nucleic acid molecules comprise prokaryotic and/or eukaryotic mRNA.

The present invention claims priority to U.S. Provisional Patent Application Ser. Nos. 60/651,409, filed Feb. 9, 2005 and 60/685,367 filed May 27, 2005, each of which is herein incorporated by reference in its entirety.

### FIELD OF THE INVENTION

The present invention relates to compositions and methods employing 5′-phosphate-dependent nucleic acid exonucleases. In particular, the present invention provides kits and methods for employing 5′-phosphate-dependent nucleic acid exonucleases for selective enrichment, isolation and amplification of a particular set of desired nucleic acid molecules from samples that also contain undesired nucleic acid molecules for a variety of uses. In preferred embodiments, the desired nucleic acid molecules comprise prokaryotic and/or eukaryotic mRNA.

### BACKGROUND OF THE INVENTION

With the enormous increase in the amount of bacterial genome sequence information over the last several years and the complete sequencing of a number of microbial genomes, the molecular biology field has entered a post-genomic era (Pang et al., Microbiol. Immunol., 48:91, 2004). The investigation of gene expression at the transcriptional level is of basic interest in microbial ecology and pathogenicity studies. This investigation will bridge the gap between structural and functional diversity. Expression analysis is being used to identify gene functions and metabolic pathways in many organisms, including humans, yeast, Drosophila, mice, and bacteria.

A major challenge in prokaryotic expression analysis is the preparation and analysis of prokaryotic mRNA. Oligo(dT) selection for poly(A) tails has long been used for isolating mRNAs from eukaryotic sources. However, the lack of relatively stable poly(A) tails, and their short half-lives and low quantity for bacteria make isolation of bacterial mRNA difficult (Adel et al., Nature. Biotechnol. 18:679, 2000) and Coller et al., Proc. Natl. Acad. Sci. U.S.A. 97:3260, 2000). Thus, isolation of mRNA from bacteria has been vitally important, but difficult. There has been an urgent need to develop rapid and simple methods to purify mRNA from bacteria. Several attempts are described below. However, none have provided sufficient solutions to the problem.

Wendisch et al. (Anal. Biochem. 290:205, 2001); U.S. Pat. No. 6,242,189, herein incorporated by reference in their entireties) developed a method to purify the whole population of cellular mRNAs by polyadenylation with E. coli poly(A) polymerase in crude cell extracts obtained by mechanical lysis. However, this method does not select for only mRNA, so other RNA molecules can be polyadenylated in addition to mRNA.

Affymetrix Inc. (Rosenow et al., Nucleic Acids Res. 29(22):e112, 2001) obtained enriched mRNA from E. coli with a series of enzymatic steps that specifically eliminate the 16S and 23S rRNA species in the total RNA. Reverse transcriptase and primers specific for 16S and 23S rRNA are used to synthesize complementary DNAs. Then rRNA is removed enzymatically by treatment with RNase H, which specifically digests RNA within an RNA:DNA hybrid. The cDNA molecules are then removed by DNase I digestion and the enriched mRNA is purified on QIAGEN RNeasy columns. The enriched mRNA will contain mRNAs, tRNAs, 5S rRNA, and other small RNAs. This method is complex and difficult to handle, and introduces the risk of mRNA losses due to mispriming of mRNA with rRNA primers.

Ambion Company developed the MICROBE EXRESS Bacterial mRNA Purification Kit that employs a modified capture hybridization approach, to remove abundant 16S and 23S ribosomal RNAs (rRNA) from purified total RNA and enrich bacterial mRNA. Briefly, purified RNA is incubated with the Capture Oligonucleotide Mix in Binding Buffer. Magnetic beads, derivatized with an oligonucleotide that hybridizes to the capture oligonucleotide, are then added to the mixture and allowed to hybridize. The magnetic beads, with 16S and 23S rRNAs attached, are pulled to the side of the tube with a magnet. The enriched RNA in the supernatant is removed and precipitated with ethanol. The enriched mRNA will contain mRNAs, tRNAs, 5S rRNA, and other small RNAs. The process is somewhat tedious and cumbersome, and cannot be applied to all species of bacteria. U.S. Pat. Appln. No. 2003/0175709 likewise describes a method for depleting unwanted RNA species using capture probes that hybridize to bridging oligonucleotides configured to bind to capture beads and target sequences.

Pang et al., supra, demonstrate that magnetic capture-hybridization methods may be used for the purification of bacterial mRNAs, using biotin-labeled oligonucleotides as capture probes specific for 5S, 16S and 23S rRNA of bacteria. Ribosomal RNAs hybridize to biotin-labeled oligonucleotide capture probes that are fixed to streptavidin-coated paramagnetic beads. The mRNA remains in the supernatant and is recovered by ethanol precipitation. While this method enriched mRNA, improvements in efficiency are needed.

Thus, the art is in need of improved compositions and methods that provide high efficiency and ease of use for the enrichment and purification of bacterial mRNA for a variety of applications. What is needed are rapid and simple methods for isolation and purification of bacterial mRNA that avoid the tedious and cumbersome use of magnetic beads or purification columns, and/or the synthesis of ribosomal cDNA and subsequent RNase H digestion.

What is further needed in the art are compositions and methods for preparation of bacterial mRNA, whereby said compositions and methods remove 3′ mRNA fragments that do not have the 5′-end of primary mRNA transcripts (such as 3′ mRNA fragments resulting from exposure of primary mRNA transcripts to a ribonuclease or to physical forces such as shearing).

What is further needed are compositions and methods to prepare cDNA from bacterial mRNA and to amplify bacterial mRNA for gene expression studies such as, but not limited to, for microarray analyses. Moreover, what is needed are compositions and methods for preparation of cDNA from bacterial mRNA, whereby the cDNA is synthesized from full-length mRNA, meaning from mRNA sequences that are not truncated at the 5′-end or the 3′-end.

What is further needed are compositions and methods to isolate bacterial mRNA, to prepare cDNA from bacterial mRNA, and to amplify bacterial mRNA for gene expression studies from samples in which the bacteria are associated with cells of one or more other prokaryotic and/or eukaryotic organisms. By way of example, but not of limitation, what is needed are compositions and methods for purifying bacterial mRNA, making bacterial cDNA, and amplifying bacterial mRNA from Rhizobium nitrogen-fixing bacteria in the roots of legumes, from biofilms such as in the human mouth, or from pathogenic bacteria or mycoplasma in association with a plant, animal, human, or fungal host. Moreover, what is needed are improved compositions and methods for isolating mRNA, making cDNA, and amplifying mRNA from both pathogen (or symbiont) and host cells at the same time in order to simultaneously study gene expression in both organisms that are associated in pathogen-host (or symbiotic) interactions (or even to simultaneously study gene expression in multiple bacterial species associated in biofilms of any type, or in multiple cells of a single organism or of multiple organisms in any type of association).

What is also needed are compositions and methods that enable synthesis of cDNA and amplification of mRNA from bacteria that are difficult to grow in cell culture.

What is also needed in the art are improved compositions and methods to purify eukaryotic mRNA. What is needed are rapid and simple methods for isolation and purification of eukaryotic mRNA that avoid the tedious and cumbersome use of magnetic beads, purification columns, or membranes that bind polyadenylated nucleic acids (e.g., with oligo(dT)). Moreover, what is needed are compositions and methods for preparation of eukaryotic mRNA, whereby said compositions and methods remove mRNA fragments that do not have the 5′-end of a full-length mRNA transcript (such as 3′ mRNA fragments resulting from exposure of the mRNA to a ribonuclease or to physical forces such as shearing and/or heat).

What is further needed are improved compositions and methods to prepare cDNA from eukaryotic mRNA and to amplify eukaryotic mRNA for gene expression studies such as, but not limited to, for microarray analyses. Moreover, what is needed are compositions and methods for preparation of cDNA from eukaryotic mRNA, whereby the cDNA is synthesized from full-length mRNA, meaning from mRNA sequences that are not truncated at the 5′-end or the 3′-end. What is further needed are improved compositions and methods for isolating mRNA, making cDNA, and amplifying mRNA from eukaryotes in a high-throughput manner.

What is also needed are improved kits and methods for analyzing gene expression in biological samples containing eukaryotic mRNA or both eukaryotic and prokaryotic mRNA. For example, it can be very difficult to obtain mRNA, make cDNA, and amplify mRNA in biological samples that contain degraded RNA, such as, but not limited to slides of formalin-fixed paraffin-embedded (“FFPE”) tissue sections. Samples of cells from which it is desired to profile gene expression can be obtained from such sections by various methods known in the art. One method that can be used to obtain sample of cells from such tissue sections is laser capture, such as but not limited to, laser capture microdissection, using methods known in the art. Instruments for laser capture are available commercially from Arcturus, PALM, and other sources. Methods are also known in the art for isolating total RNA from samples from FFPE tissue sections. One such method that can be used is described by Acturus in the product literature for their Paradise™ Kit. Other methods are also known in the art that can be used.

However, once total RNA is obtained, it can be difficult to obtain good quality mRNA for gene expression analysis by methods known in the art, such as but not limited to, analysis by microarrays. In part, this is true because many methods rely on use of oligo(dT) resins or membranes to isolate poly(A) containing RNA, which is a common method for isolating polyadenylated mRNA. The problem is that much of the mRNA may be degraded into fragments that do not have a poly(A) tail and will not be isolated using such oligo(dT) columns or membranes. Also, the mRNA fragments that have a poly(A) tail and therefore are obtained using the oligo(dT) binding method may not contain the sequences that are complementary to many of the oligos on an array or microarray. Therefore, the gene expression analysis can be difficult to interpret. It would be desirable to obtain mRNA samples more easily, particularly for making cDNA, for RNA amplification and other methods of amplification, and especially, for obtaining better and more informative gene expression data than current methods.

### SUMMARY OF THE INVENTION

The present invention relates to compositions and methods employing 5′-phosphate-dependent nucleic acid exonucleases. In particular, the present invention provides kits and methods employing 5′-phosphate-dependent nucleic acid exonucleases for selective enrichment, isolation and amplification of a particular set of desired nucleic acid molecules from samples that also contain undesired nucleic acid molecules for a variety of uses. In preferred embodiments, the desired nucleic acid molecules comprise prokaryotic and/or eukaryotic mRNA.

The present invention comprises methods for using a 5′ exoribonuclease for a number of applications related to biological research on gene expression, diagnostics, and human and animal therapeutics.

One preferred method of the invention comprises a method for enriching for an RNA having a 5′-triphosphate or a 5′-cap in a biological sample comprising prokaryotic RNA, eukaryotic RNA or both prokaryotic and eukaryotic RNA and at least one undesired nucleic acid, the method comprising treating the sample with purified 5′ exoribonuclease under conditions in which the 5′ exoribonuclease is active and for sufficient time so that the undesired nucleic acid is digested and the sample is enriched for RNA having a 5′-triphosphate or a 5′-cap.

In different embodiments of this method, the RNA having a 5′-triphosphate or a 5′-cap is selected from the group consisting of: (i) prokaryotic mRNA; (ii) eukaryotic mRNA, including polyadenylated and non-polyadenylated eukaryotic mRNA; (iii) a mixture of both prokaryotic and eukaryotic mRNA; (iv) eukaryotic snRNA; (v) eukaryotic pre-micro RNA; and (vi) prokaryotic or eukaryotic primary RNA transcripts of unknown function.

Another preferred embodiment of this method comprises treating the biological sample with a polynucleotide kinase to phosphorylate the RNA having 5′-hydroxyl groups prior to treating the sample with purified 5′ exoribonuclease.

In one embodiment of the method, the RNA having a 5′-triphosphate or a 5′-cap comprises eukaryotic mRNA or both prokaryotic and eukaryotic mRNA, and the method additionally comprises binding the RNA to an oligo(dT) or oligo(dU) resin, membrane or other surface to which oligo(dT) or oligo(dU) is attached and eluting the bound RNA so as to obtain a solution containing polyadenylated eukaryotic mRNA.

In another embodiment of the method, the RNA having a 5′-triphosphate or a 5′-cap comprises prokaryotic mRNA, eukaryotic mRNA or both prokaryotic and eukaryotic mRNA and the undesired nucleic acid comprises prokaryotic rRNA, eukaryotic rRNA or both prokaryotic and eukaryotic rRNA of a size having a svedburg unit greater than about 10S. In specific embodiments of this method the rRNA is selected from the group consisting of prokaryotic rRNA having a svedburg unit of about 16S or 23S and eukaryotic rRNA having a svedburg unit of about 18S, 26S or 28S.

Another embodiment of the invention is a method wherein the RNA having a 5′-triphosphate or a 5′-cap comprises prokaryotic mRNA, eukaryotic mRNA, or both prokaryotic and eukaryotic mRNA, and wherein the method additionally comprises precipitation of said mRNA having a 5′-triphosphate or a 5′-cap with a solution of LiCl and ethanol at concentrations and under conditions in which tRNA and 5S rRNA are not precipitated. In some embodiments of this method, the RNA having a 5′-triphosphate or a 5′-cap comprises prokaryotic mRNA, eukaryotic mRNA, or both prokaryotic and eukaryotic mRNA and the undesired nucleic acid comprises prokaryotic rRNA, eukaryotic rRNA or both prokaryotic and eukaryotic rRNA of a size having a svedburg unit greater than about 10S. In specific embodiments, the rRNA is selected from the group consisting of prokaryotic rRNA having a svedburg unit of about 16S or 23S and eukaryotic rRNA having a svedburg unit of about 18S, 26S or 28S.

Still another method of the invention that uses RNA obtained following 5′ exoribonuclease treatment comprises: (i) contacting the sample enriched for RNA having a 5′-triphosphate or a 5′-cap with a poly(A) polymerase under conditions so that polyadenylated RNA is obtained; and (ii) contacting the polyadenylated RNA with an oligo(dT)-containing primer and an RNA-dependent DNA polymerase under conditions that cDNA complementary to the RNA is obtained. In some specific embodiments, the oligo(dT)-containing primer comprises a T7-type RNA polymerase promoter selected from the group consisting of T7 RNA polymerase, T3 RNA polymerase, and SP6 RNA polymerase. In one embodiment of this method, the biological sample is treated with a polynucleotide kinase to phosphorylate the RNA having 5′-hydroxyl groups prior to treating the sample with purified 5′ exoribonuclease. In some other specific embodiments of this method, the RNA having a 5′-triphosphate or a 5′-cap comprises prokaryotic mRNA, eukaryotic mRNA, or both prokaryotic and eukaryotic mRNA and the undesired nucleic acid comprises prokaryotic rRNA, eukaryotic rRNA or both prokaryotic and eukaryotic rRNA of a size having a svedburg unit greater than about 10S. In specific embodiments, the rRNA is selected from the group consisting of prokaryotic rRNA having a svedburg unit of about 16S or 23S and eukaryotic rRNA having a svedburg unit of about 18S, 26S or 28S.

In still another method, wherein the biological sample comprises eukaryotic RNA, and wherein the method additionally comprises contacting the sample enriched for RNA having a 5′-triphosphate or a 5′-cap with an oligo(dT)-containing primer and an RNA-dependent DNA polymerase under conditions that cDNA complementary to the RNA is obtained. In some specific embodiments, the oligo(dT)-containing primer comprises a T7-type RNA polymerase promoter selected from the group consisting of T7 RNA polymerase, T3 RNA polymerase, and SP6 RNA polymerase. In some of these embodiments, the method additionally comprises contacting the cDNA complementary to the RNA with one or more DNA or RNA primers and a DNA polymerase and incubating under conditions so as to obtain double-stranded cDNA having a functional promoter for the T7-type RNA polymerase and synthesizing RNA therefrom.

In still other embodiments, the method additionally comprises contacting the cDNA complementary to the RNA with an oligonucleotide sequence tag template and a DNA polymerase and incubating under conditions so as to obtain cDNA having a sequence tag on it the 3′-end. In some embodiments, the method additionally comprises contacting the cDNA having the sequence tag on its 3′-end with a DNA polymerase and an oligonucleotide primer, wherein the either the sequence tag or the oligonucleotide primer, or the combination of both the sequence tag and the oligonucleotide primer encodes the complete sequence of a T7-type RNA polymerase promoter, and incubating under conditions wherein double-stranded cDNA having a functional T7-type RNA polymerase promoter is obtained. In those cases, the method additionally comprises contacting the cDNA with the T7-type RNA polymerase under conditions so as to synthesize RNA using said T7-type RNA polymerase promoter. In some other specific embodiments of these methods, the biological sample is treated with a polynucleotide kinase to phosphorylate the RNA having 5′-hydroxyl groups prior to treating the sample with purified 5′ exoribonuclease.

In preferred embodiments, the compositions, kits, and methods of the present invention employ Xrn1p/5′ Exoribonuclease I and functional variants, homologues, and equivalents. Xrn1p/5′ exoribonuclease I is a magnesium-dependent, 5′-to-3′, processive exoribonuclease that acts preferentially on RNA substrates with a 5′ phosphate (Stevens, Biochem. Biophys. Res. Commun. 81:656, 1978); Stevens, Biochem. Biophys. Res. Commun. 86:1126, 1979), Stevens, J. Biol. Chem. 255:3080, 1980); and Stevens and Maupin, Nucleic Acids Res. 15:695, 1987). Nucleic acid molecules with a cap, a triphosphate group, or an hydroxyl group on their 5′-termini (or 5′-ends) are not substantially digested by the enzyme. The exoribonuclease I is not inhibited by proteinaceous RNase inhibitors such as RNASIN (Promega, Madison, Wis.) or PRIME RNase Inhibitor (Eppendorf, Brinkmann, Westbury, N.Y.). In some embodiments of the present invention, the 5′-phosphate-dependent nucleic acid exonuclease is obtained as described by Stevens (J. Biol. Chem. 255:3080, 1980). In some other embodiments, the 5′-phosphate-dependent nucleic acid exonuclease is obtained by cloning a gene for an exonuclease, such as but not limited to a Saccharomyces Xrn I gene, in a vector, expressing the gene in a host cell, and purifying the expressed protein from cultures of said host cells. For example, but without limitation, in some embodiments, the 5′-phosphate-dependent nucleic acid exonuclease is obtained from recombinant yeast by following the protocol of Johnson and Kolodner (J. Biol. Chem. 266:14046, 1991). In other embodiments, the 5′-phosphate-dependent nucleic acid exonuclease is obtained by cloning the gene for an exonuclease in a vector, expressing the gene in a heterologous host cell, such as but not limited to an E. coli host cell, and purifying the expressed protein from cultures of said host cells. Such a composition from recombinant source finds use for more efficient and less expensive production, as well as a higher purity and more consistent quality of 5′-phosphate-dependent nucleic acid exonuclease for use in the methods and kits of the present invention. The expression vector may further express other sequences that assist in the purification, expression, detection, or use of the 5′-phosphate-dependent nucleic acid exonucleases.

The exonucleases of the present invention find use in a number of applications including, but not limited to: analysis of the 5′ ends of RNA molecules; removal of ribosomal RNA from RNA preparations; enrichment of prokaryotic and/or eukaryotic mRNA; enrichment of mixtures of prokaryotic and/or eukaryotic mRNA; preparation of prokaryotic and/or eukaryotic mRNA for cDNA synthesis; synthesis of amplified antisense or sense RNA by preparing prokaryotic mRNA using exoribonuclease, then polyadenylating the RNA with poly(A) polymerase, then making cDNA joined to an RNA polymerase promoter, and transcribing the cDNA from the promoter in vitro; enrichment of 5′-capped or 5′-triphosphorylated small nuclear RNAs (snRNA); removal of 5′-phosphorylated splice template oligos (or template switch oligos) and other oligos (e.g., ligation splints) from in vitro transcription reactions, including antisense or sense RNA amplification reactions, in order to reduce nonspecific background transcription in RNA amplification and other reactions; removal of linear RNA from circular RNA preparations; removal of linear first-strand cDNA molecules from circular first-strand cDNA molecules, diagnostic applications; and the like.

For example, in some embodiments, the present invention provides a method for isolating a nucleic acid molecule of interest in a mixture of nucleic acids, comprising the steps of: a) providing a 5′-to-3′ exoribonuclease and a biological sample comprising a mixture of nucleic acids, said mixture of nucleic acids comprising a nucleic acid molecule of interest (i.e., a desired nucleic acid) and an undesired nucleic acid having a 5′-phosphate; and b) exposing the 5′-to-3′ exoribonuclease to the biological sample under conditions such that the exoribonuclease digests the undesired nucleic acid so as to enrich the sample for the nucleic acid molecule of interest. The present invention is not limited by the nature of the 5′-phosphate-dependent nucleic acid exonuclease. In preferred embodiments, the 5′-phosphate-dependent nucleic acid exonuclease includes, but is not limited to, Xrn1p/5′ exoribonuclease 1, fragments of Xrn1p/5′ exoribonuclease 1 (e.g., that have exoribonuclease activity), and Xrn1p/5′ exoribonuclease 1 variants (e.g., containing conserved amino acid changes, truncations, chimeras, etc.). A 5′ exonuclease of the present invention comprises any 5′-exonuclease that has greater than 20-fold more 5′-to-3′ exonuclease activity for a single-stranded RNA substrate that has a 5′-monophosphorylated terminus than for the same RNA substrate that has a 5′-triphosphorylated or 5′-capped terminus. That is, a 5′ exonuclease of the present invention comprises any 5′-to′3′ exonuclease that has a relative activity in digesting a particular defined-sequence single-stranded RNA substrate with a 5′-triphosphate or a 5′-cap that is less than 5% of the activity of an RNA substrate having the same sequence but with a 5′-monophosphate. Enzyme activity of a 5′ exonuclease of the invention can be measured using a number of different methods. Without limitation, suitable methods that can be used for assaying activity and determining relative activity using RNA substrates with a 5′-triphosphate, a 5′-cap, or a 5′-monophosphate are described by Stevens and Poole (J. Biol. Chem. 270: 16063, 1995).

The present invention is also not limited by the nature of the sample. Samples include, but are not limited to, cell lysates, provided that nucleases that can affect nucleic acids of interest are removed or inhibited in said lysates, mixtures of nucleic acids (e.g., unpurified, partially purified, etc.), environmental samples, etc. Samples can comprise nucleic acids from a 1-cell organism, or if an organism comprises multiple cells, from one or more cells. Samples comprising nucleic acids from multiple cells can comprise cells from one or more types of cells, including cells from different organisms, and/or different cells from the same organism. The present invention finds use with prokaryotic nucleic acid, with eukaryotic nucleic acid, or with a mixture of both eukaryotic and prokaryotic nucleic acid.

In some preferred embodiments of the present invention, the sample comprises (or contains) RNA. A sample of the invention that comprises or contains RNA can contain total RNA from any source, which RNA can be obtained using, for example, a MasterPure RNA Purification Kit, an ArrayPure Nano-Scale RNA Purification Kit, or a MasterPure Yeast RNA Purification Kit (all of which are from EPICENTRE), or using a kit from another commercial source, or using a “home-brew” method known in the art. Alternatively, in some embodiments a sample of the invention that comprises or contains RNA can contain a subfraction of total RNA obtained by any method known in the art, such as, but without limitation, a subfraction based on size (e.g., by purification on an agarose or polyacrylamide gel, or by column purification, including by HPLC), or a subfraction obtained by salt precipitation (e.g., using precipitation with 0.5-2.5 M LiCl (Barlow, J J et al., Biochem. Biophys. Res. Comm. 13: 61, 1963); Cathala, G et al, DNA 2: 329, 1983) or 2.5 M ammonium acetate). In some embodiments, a sample comprising RNA can also contain DNA. In some preferred embodiments, the nucleic acid molecules of interest comprise mRNA having a 5′-triphosphate or a 5′-cap. However, the nucleic acid molecule of interest (or desired nucleic acid) can comprise any nucleic acid molecule that is resistant to digestion by the 5′-phosphate-dependent nucleic acid exonuclease used, including, but not limited to nucleic acid molecules having a 5′-triphosphate or a 5′-cap, a 5′-hydroxyl group, or circularized nucleic acid molecules. The undesired nucleic acid can comprise any nucleic acid molecule that is digested by the 5′-phosphate-dependent nucleic acid exoribonuclease used, including, but not limited to nucleic acid molecules having a 5′-monophosphate or a 5′-diphosphate group, which are substrates for the enzyme. In some preferred embodiments, the undesired nucleic acid comprises rRNA that is greater than approximately 200 nucleotides (e.g., without limitation, containing each of 16S and 23S prokaryotic rRNA or 18S and 28S human, mouse, drosophila, or Xenopus rRNA or 18S and 26S yeast rRNA).

In some preferred embodiments, little or no detectable level of the desired nucleic acid molecule of interest is digested by the 5′-phosphate-dependent nucleic acid exonuclease. However, the invention is not limited by the amount of the desired nucleic acid molecule of interest that is digested so long as a substantial amount of the undesired nucleic acid is digested and sufficient amount of the desired nucleic acid remains for the intended purpose, which can vary for different purposes. For example, less of the desired RNA may be needed from some samples and/or applications (e.g., for RNA amplification of mRNA from total RNA in large samples) than are needed from other samples and/or applications (e.g., preparing a cDNA library from only a small number of cells). In preferred embodiments, a substantial amount of the undesired nucleic acid is digested by the 5′ exonuclease of the invention, which means that at least 50% (e.g., 60%, 70%, 80%, 90%, 95%, 98%, 99%) of the starting amount (i.e., the amount present prior to treatment with the exonuclease) of the undesired nucleic acid is digested.

In some preferred embodiments, the present invention provides a method for isolating prokaryotic mRNA (e.g., to conduct gene expression analysis) comprising the steps of: a) providing a sample comprising RNA from a prokaryotic organism, said sample containing desired mRNA having a 5′-triphosphate group and at least one undesired nucleic acid; and a 5′-phosphate-dependent nucleic acid exonuclease; b) treating the sample with the 5′-phosphate-dependent nucleic acid exonuclease under conditions such that said undesired nucleic acid is substantially digested and said desired mRNA is not digested; and c) recovering the mRNA (e.g., for additional analysis or use). In some embodiments, the 5′-phosphate-dependent nucleic acid exonuclease used in the method is provided in a kit with buffers, instructions, and optionally, with appropriate control samples, and the like.

In some preferred embodiments, the present invention provides a method for isolating prokaryotic mRNA molecules that have the 5′-ends of primary mRNA transcripts (e.g., to conduct gene expression analysis) comprising the steps of: a) providing a sample comprising RNA from a prokaryotic organism, said sample containing desired mRNA having a 5′-triphosphate group and at least one undesired nucleic acid; a polynucleotide kinase; and a 5′-phosphate-dependent nucleic acid exonuclease; b) treating the sample with the polynucleotide kinase in the presence of ATP and under conditions such that nucleic acid molecules having an hydroxyl group at their 5′-termini are phosphorylated; c) treating the sample with the 5′-phosphate-dependent nucleic acid exonuclease under conditions such that RNA molecules having a 5′-monophosphate group are substantially digested; and d) recovering the mRNA (e.g., for additional analysis or use). In some embodiments, the 5′-phosphate-dependent nucleic acid exonuclease used in the method is provided in a kit with polynucleotide kinase (e.g., T4 polynucleotide kinase), ATP, buffers, instructions, and optionally, with appropriate control samples, and the like.

In some embodiments, the present invention provides methods and kits for producing cDNA copies of prokaryotic mRNA (e.g., for making cDNA libraries). For example, the present invention provides a method for preparing prokaryotic cDNA, comprising the steps of a) providing a sample comprising RNA from a prokaryotic organism, said sample containing desired mRNA having a 5′-triphosphate group and at least one undesired nucleic acid; and a 5′-phosphate-dependent nucleic acid exonuclease; b) contacting the sample with the 5′-phosphate-dependent nucleic acid exonuclease under conditions such that undesired nucleic acids in the sample are substantially digested but mRNA molecules in the sample having a 5′-triphosphate are not digested; and c) producing cDNA copies of the mRNA. The present invention is not limited by the manner in which the cDNA copies are produced.

In some preferred embodiments, the method of making cDNA copies comprises: i) contacting the mRNA molecules with a poly(A) polymerase to produce mRNA molecules having a 3′-poly(A) tail; and ii) contacting the mRNA molecules having a 3′-poly(A) tail with a primer that anneals to said tail (e.g., an oligo(dT)-containing primer) and extending the primer with an RNA-dependent DNA polymerase under conditions whereby cDNA is obtained. Kits of the invention for conducting such methods may have any one or more reagents (e.g., primers), control samples, buffers, etc. useful in the method. For example, preferred kits comprise: a) a 5′-phosphate-dependent nucleic acid exonuclease; b) a poly(A) polymerase (e.g., without limitation, an E. coli poly(A) polymerase encoded by the pcnB gene); and c) an RNA-dependent DNA polymerase (e.g., an AMV reverse transcriptase; an MMLV reverse transcriptase; SuperScript I, SuperScript II, SuperScript III, or AMV ThermoScript reverse transcriptase (INVITROGEN); or MonsterScript reverse transcriptase (EPICENTRE).

In some other embodiments, the method of making cDNA copies comprises adding a sequence tag template to the 3′-end of the mRNA molecules using a method as described in U.S. Patent Application No. 2005/0153333, incorporated herein by reference. Thus, one embodiment for making cDNA copies of mRNA molecules comprises: i) contacting the mRNA molecules with a mixture of tagging oligonucleotides, each tagging oligonucleotide having a 5′-portion comprising the same sequence tag template, a 3′-portion comprising a random sequence and a blocked 3′ terminus, under conditions in which one of the tagging oligonucleotides anneals with each mRNA molecule and the mRNA molecules are extended with a nucleic acid polymerase to form mRNA molecules having a sequence complementary to the sequence tag template on their 3′-termini; and ii) contacting the mRNA molecules having a sequence complementary to the sequence tag template on their 3′-termini with a primer that anneals to said sequence that is complementary to the sequence tag template and extending the primer with an RNA-dependent DNA polymerase under conditions whereby cDNA is obtained. In some aspects of this embodiment, the nucleic acid polymerase used to extend the mRNA molecules using the sequence tag template as a template in step (i) is the same enzyme as the RNA-dependent DNA polymerase used to obtain cDNA in step (ii), wherein, in other aspects of this embodiment, the nucleic acid polymerase used to extend the mRNA molecules using the sequence tag template as a template in step (i) is different than the RNA-dependent DNA polymerase used to obtain cDNA in step (ii). Kits for synthesizing cDNA according to the embodiment of the invention comprise: a) tagging oligonucleotides; b) a primer that anneals to the sequence that is complementary to the sequence tag template; and c) an RNA-dependent DNA polymerase (e.g., an AMV reverse transcriptase; an MMLV reverse transcriptase; SuperScript I, SuperScript II, SuperScript III, or AMV ThermoScript reverse transcriptase (INVITROGEN); or MonsterScript reverse transcriptase (EPICENTRE)); and in aspects of this embodiment in which the nucleic acid polymerase that extends the 3′-termini of the mRNA molecules using the sequence that is complementary to the sequence tag template of the tagging oligonucleotide as a template is different from the RNA-dependent DNA polymerase used to obtain cDNA, the kit additionally comprises said nucleic acid polymerase for extending the 3′-termini of the mRNA molecules.

In some preferred embodiments, the present invention provides methods and kits for producing cDNA copies of full-length prokaryotic mRNA (e.g., for making full-length cDNA libraries). For example, the present invention provides a method for preparing prokaryotic cDNA, comprising the steps of a) providing a sample comprising RNA from a prokaryotic organism; a polynucleotide kinase; and a 5′-phosphate-dependent nucleic acid exonuclease; b) contacting the sample with the polynucleotide kinase in the presence of ATP and under conditions such that nucleic acid molecules having an hydroxyl group at their 5′-termini are phosphorylated; c) contacting the sample treated with the polynucleotide kinase with the 5′-phosphate-dependent nucleic acid exonuclease under conditions such that undesired nucleic acids in the sample are digested but mRNA molecules in the sample having a 5′-triphosphate are not digested; and d) producing cDNA copies of the mRNA. The present invention is not limited by the manner in which the cDNA copies are produced.

In some preferred embodiments, the method of making cDNA copies comprises: i) contacting the mRNA molecules with a poly(A) polymerase to produce mRNA molecules having a 3′-poly(A) tail; and ii) contacting the mRNA molecules having a 3′-poly(A) tail with a primer that anneals to said tail and extending the primer with an RNA-dependent DNA polymerase under conditions whereby cDNA is obtained. Kits for conducting such methods may have any one or more reagents (e.g., oligo(dT) primer and/or other primers), control samples, ATP, buffers, etc. useful in the method. For example, preferred kits comprise: a) a 5′-phosphate-dependent nucleic acid exonuclease; b) a polynucleotide kinase (e.g., T4 polynucleotide kinase); c) a poly(A) polymerase; and d) an RNA-dependent DNA polymerase. In some preferred embodiments, the desired nucleic acid molecules comprise both prokaryotic and eukaryotic mRNA. In some preferred kits in which the RNA in the sample is of sufficient quality for the intended purpose, meaning that a sufficient amount of the RNA comprises the desired mRNA with a 5′-triphosphate or 5′-cap for the intended purpose (which amount can vary for different purposes), the step comprising contacting the sample with the polynucleotide kinase in the presence of ATP and under conditions such that nucleic acid molecules having an hydroxyl group at their 5′-termini are phosphorylated can be omitted and a polynucleotide kinase is not needed in a preferred kit for this method, or if it is included in the kit, the polynucleotide kinase step can be omitted. The invention also envisions that this step can be omitted when a sample is rare or limited in amount and it is desirable to obtain the maximum amount of mRNA and/or cDNA, including degraded portions of mRNA and/or cDNA molecules, for the intended purposes.

Similar methods and kits are provided that are useful for amplifying prokaryotic mRNA. For example, the present invention provides a method for amplifying prokaryotic mRNA, comprising the steps of a) providing a sample comprising RNA from a prokaryotic organism; and a 5′-phosphate-dependent nucleic acid exonuclease; b) contacting the sample with the 5′-phosphate-dependent nucleic acid exonuclease under conditions such that undesired nucleic acids in the sample are substantially digested but mRNA molecules in the sample having a 5′-triphosphate are not digested; and c) amplifying the mRNA. Similar methods and kits are provided that are useful for amplifying prokaryotic mRNA, whereby the methods and kits result in enrichment of sequences corresponding to the 5′ portions of mRNA. For example, the present invention provides a method for amplifying prokaryotic mRNA, comprising the steps of a) providing a sample comprising RNA from a prokaryotic organism; a polynucleotide kinase; and a 5′-phosphate-dependent nucleic acid exonuclease; b) contacting the sample with the polynucleotide kinase in the presence of ATP and under conditions such that nucleic acid molecules having an hydroxyl group at their 5′-termini are phosphorylated; c) contacting the sample with the 5′-phosphate-dependent nucleic acid exonuclease under conditions such that undesired nucleic acids in the sample are substantially digested but mRNA molecules in the sample having a 5′-triphosphate are not digested; and d) amplifying the mRNA. The present invention is not limited by the manner in which the mRNA is amplified. In some embodiments, the mRNA amplified by a method comprising: i) contacting said mRNA molecules with a poly(A) polymerase to produce mRNA molecules having a 3′-poly(A) tail; ii) contacting said mRNA molecules having a 3′-poly(A) tail with a primer that anneals to said tail and extending said primer with an RNA-dependent DNA polymerase under conditions whereby cDNA that is joined to an RNA polymerase promoter sequence is obtained (using any number of methods known in the art; methods are known for joining the promoter to either the 5′-end of the cDNA or the 3′-end of the cDNA, resulting in synthesis of amplified antisense or sense RNA, respectively, by subsequent in vitro transcription); iii) producing double stranded cDNA; and iv) contacting said double-stranded cDNA comprising said promoter sequence with an RNA polymerase that binds to said promoter and synthesizes RNA therefrom under conditions whereby RNA corresponding to mRNA in the biological sample is synthesized. Some other embodiments of the present invention use a single-stranded first-strand cDNA template that is functionally joined at either the 3′-end or the 5′-end to a single-stranded promoter that is recognized by an RNA polymerase, such as MiniV RNA polymerase, that can synthesize RNA in vitro therefrom; in these embodiments, the method does not comprise a step for synthesis of double-stranded cDNA. Kits for conducting such methods may have any one or more reagents (e.g., primers), control samples, buffers, etc. useful in the method. For example, preferred kits comprise: a) a 5′-phosphate-dependent nucleic acid exonuclease; c) an RNA-dependent DNA polymerase and c) an RNA polymerase (e.g., T7, T3, and SP6 RNA polymerases). Other kits that use single-stranded templates and promoters use an RNA polymerase that synthesize RNA in vitro therefrom, such as MiniV RNA polymerase. Other preferred kits further comprise: a) a polynucleotide kinase, such as but not limited to T4 polynucleotide kinase, and ATP; and b) a poly(A) polymerase, such as but not limited to a poly(A) polymerase encoded by an E. coli pcnB gene.

In other preferred embodiments, the present invention provides a method for enriching for or isolating mRNA from eukaryotes, comprising the steps of a) providing a 5′-phosphate-dependent nucleic acid exonuclease; and a biological sample comprising a mixture of nucleic acids, said mixture of nucleic acids comprising mRNA of interest and an undesired nucleic acid having a 5′-phosphate; and b) contacting the 5′-phosphate-dependent nucleic acid exonuclease to the biological sample under conditions such that the exonuclease substantially digests the undesired nucleic acid so as to enrich said sample for the mRNA of interest. Thus, the present invention provides alternative kits and methods for isolating eukaryotic mRNA that do not require binding to oligo(dT) cellulose or other oligo(dT) or oligo(du) resins or membranes. The method is particularly suitable to high-throughput methods-much more so than methods that employ binding to a resin. However, it will also be understood, based on the description of the present invention, that a 5′-phosphate-dependent exonuclease can be used to obtain a mixture of both prokaryotic and eukaryotic mRNA, and that the eukaryotic mRNA can be separated from prokaryotic mRNA in a mixture by binding polyadenylated eukaryotic mRNA to an oligo(dT) resin, as is known in the art. Still further, although most eukaryotic mRNA is believe to be polyadenylated, some eukaryotic mRNA transcripts may not have a poly(A) tail. The methods disclosed herein can be used to study eukaryotic transcripts that lack a poly(A) tail, which transcripts would not have been detected in many studies that have used oligo(dT) resins and membranes for isolation of the eukaryotic mRNA.

In some preferred embodiments, the present invention provides a method for isolating eukaryotic mRNA comprising the steps of: a) providing a sample from a eukaryotic organism comprising a mixture of nucleic acids, said mixture of nucleic acids comprising desired mRNA of interest and at least one undesired nucleic acid; and a 5′-phosphate-dependent nucleic acid exonuclease; b) treating the sample with the 5′-phosphate-dependent nucleic acid exonuclease under conditions such that the undesired nucleic acid is substantially digested and mRNA having a 5′-cap or a 5′-triphosphate group is not digested; and c) recovering the mRNA (e.g., for additional analysis or use). In some embodiments, the 5′-phosphate-dependent nucleic acid exonuclease used in the method is provided in a kit with buffers, instructions, and optionally, with appropriate control samples, and the like.

One problem in the art that complicates analyses involving mRNA, including analyses involving eukaryotic mRNA, relates to the fact that samples containing the mRNA can include mRNA molecules that are not full-length because they have been degraded: 1) by ribonucleases in the sample, which ribonucleases can be derived either from the cells from which the mRNA is derived or from contamination of the sample, such as by contamination with a ribonuclease A-type nuclease from human skin or other sources; or 2) by physical forces, such as by shearing forces or by forces due to contact of the mRNA with heat and/or metal ions, such as but not limited to Mn 2+cations. Many common ribonucleases, such as but not limited to RNase A-type ribonucleases (e.g., including those on human skin) and E. coli RNase I, are endoribonucleases that digest RNA to yield products having 5′-hydroxyl and 3′-phosphate groups. Without being bound by theory, it is believed that physical forces can yield a mixture of products having either a 5′-hydroxyl or a 5′-phosphate group. In any case, it is likely that many, if not most, of the degraded mRNA molecules in a sample will have a 5′-hydroxyl group, which means that they will be resistant to digestion with a 5′-exoribonuclease of the present invention. However, since most 5′-exoribonuclease-resistant eukaryotic mRNA molecules will have a 3′-poly(A) tail, these degraded molecules will be substrates for synthesis of oligo(dT)-primed cDNA synthesis and will also be substrates for many antisense RNA amplification methods or sense RNA amplification methods. Thus, these degraded, but 5′-exonuclease-resistant, mRNA molecules can result in a high representation of mRNA sequences corresponding to the 3′-end of the mRNA relative to mRNA sequences corresponding to the 5′-end of the mRNA, which can complicate interpretations of gene expression analyses, and the like. One embodiment of the present invention is a method for assessing the degree of RNA degradation, the method comprising: a) providing a first sample comprising a known quantity of RNA of unknown quality (i.e., of unknown degree of fragmentation or degradation) from any organism or mixture of organisms, the sample containing mRNA; a second control sample comprising a known quantity of substantially undegraded mRNA, preferably, but not necessarily, from the same organism or mixture of organisms; a polynucleotide kinase; and a 5′-phosphate-dependent exonuclease; b) treating one portion of each sample with the polynucleotide kinase in the presence of ATP and under conditions such that nucleic acid molecules having an hydroxyl group at their 5′-termini are phosphorylated; c) contacting the portion of each sample treated with the polynucleotide kinase and a second portion (of the same starting RNA quantity) of each sample that was not treated with the polynucleotide kinase with the 5′-phosphate-dependent nucleic acid exonuclease under conditions such that undesired nucleic acids in the sample having a 5′-phosphate are substantially digested but mRNA molecules in the sample having a 5′-triphosphate are not digested; and d) producing labeled cDNA copies or labeled amplified RNA of the mRNA in each portion of each sample using an oligo(dT)-containing primer; e), obtaining a ratio for each sample, the ratio having a numerator comprising the amount of labeled cDNA (or amplified RNA) obtained from the polynucleotide kinase-untreated portion and a denominator comprising the amount of labeled cDNA (or amplified RNA) obtained from the polynucleotide kinase-treated portion; and f) comparing the ratio obtained for the first sample with the ratio obtained for the second sample, whereby a higher ratio for the first sample than the ratio for the second sample indicates a higher relative degree of degradation of the RNA in the first sample compared to the second sample. This embodiment of the invention finds utility as an independent method of the invention, or more preferably, it is used as part of a method of the invention for obtaining cDNA or amplified sense or antisense RNA.

As indicated from the above discussion, it is usually desirable to increase the proportion of cDNA molecules and/or amplified sense or antisense RNA molecules that contain sequences corresponding to the 5′-end of the mRNA molecules in the sample (i.e., by removing degraded 3′ mRNA fragments) so that a higher percentage of the cDNA and/or the amplified sense or antisense RNA is made from full-length mRNA molecules. Thus, the present invention also provides a method to obtain mRNA from which the degraded mRNA molecules with a 5′-hydroxyl group have been removed, which can improve various analyses involving mRNA, including analyses that involve eukarytic mRNA, prokaryotic mRNA, or both eukaryotic and prokaryotic mRNA. One method of the present invention uses a polynucleotide kinase, such as but not limited to T4 polynucleotide kinase, in order to phosphorylate the 5′-hydroxyl groups of degraded mRNA molecules, thereby making them substrates for digestion by a 5′-exonuclease. Following digestion of the 5′-phosphorylated mRNA with a 5′-exonuclease of the invention, the remaining undigested mRNA molecules will be enriched for full-length mRNA, which will in turn result in cDNA molecules (from cDNA synthesis reactions using an oligo(dT)-containing primer) and amplified sense or antisense RNA molecules (from RNA amplification reactions) that have better representations of sequences corresponding to the 5′- and 3′-ends of mRNA in the sample. Thus, in some preferred embodiments, the present invention provides a method for isolating desired mRNA of interest comprising the steps of: a) providing a sample containing RNA from an organism, said sample comprising mRNA and at least one undesired nucleic acid; a polynucleotide kinase; and a 5′-phosphate-dependent nucleic acid exonuclease; b) treating the sample with the polynucleotide kinase in the presence of ATP and under conditions such that nucleic acid molecules having an hydroxyl group at their 5′-termini are phosphorylated; c) treating the sample with the 5′-phosphate-dependent nucleic acid exonuclease under conditions such that RNA molecules having a 5′-monophosphate group are substantially digested; and d) recovering the mRNA (e.g., for additional analysis or use). In some embodiments, the 5′-phosphate-dependent nucleic acid exonuclease used in the method is provided in a kit with polynucleotide kinase (e.g., T4 polynucleotide kinase), ATP, buffers, instructions, and optionally, with appropriate control samples, and the like.

In some embodiments, the present invention provides methods and kits for producing cDNA copies of eukaryotic mRNA (e.g., for making cDNA libraries). For example, the present invention provides a method for preparing eukaryotic cDNA, comprising the steps of a) providing a sample comprising RNA from a eukaryotic organism; and a 5′-phosphate-dependent nucleic acid exonuclease; b) contacting the sample with the 5′-phosphate-dependent nucleic acid exonuclease under conditions such that undesired nucleic acids in the sample are substantially digested but mRNA molecules in the sample having a 5′-triphosphate or a 5′-cap are not digested; and c) producing cDNA copies of the mRNA. In some preferred embodiments, the present invention provides methods and kits for producing cDNA copies of full-length eukaryotic mRNA (e.g., for making full-length cDNA libraries). For example, the present invention provides a method for preparing eukaryotic cDNA copies of full-length eukaryotic mRNA, comprising the steps of a) providing a sample comprising RNA from a eukaryotic organism; a polynucleotide kinase; and a 5′-phosphate-dependent nucleic acid exonuclease; b) contacting the sample with the polynucleotide kinase in the presence of ATP and under conditions such that nucleic acid molecules having an hydroxyl group at their 5′-termini are phosphorylated; c) contacting the sample treated with the polynucleotide kinase with the 5′-phosphate-dependent nucleic acid exonuclease under conditions such that undesired nucleic acids in the sample are substantially digested but desired mRNA molecules in the sample having a 5′-cap or a 5′-triphosphate are not digested; and d) producing cDNA copies of the mRNA. The present invention is not limited by the manner in which the cDNA copies are produced. In some preferred embodiments, the method of making cDNA copies comprises contacting the mRNA molecules with a primer having an oligo(dT) sequence in its 3′-end portion under conditions such that the primer anneals with the mRNA, and extending the primer with an RNA-dependent DNA polymerase under conditions whereby cDNA is obtained. In preferred embodiments, an RNA-dependent DNA polymerase enzyme and suitable reaction conditions are used so that the amount of full-length cDNA is maximized. Suitable enzymes and reaction conditions for this purpose are known in the art. For example, but without limitation, U.S. Pat. Nos. 5,962,271; and 5,962,272 describe one method to obtain increased amounts of full-length cDNA, which can be used together with the methods of the present invention. However, the invention is not limited with respect to enzymes or reaction conditions, and any RNA-dependent DNA polymerase and reaction conditions that accomplish the intended purpose can be used in a method or kit of the present invention. Kits for conducting such methods of the present invention may have any one or more reagents (e.g., an oligo(dT)-containing primer and/or other primers), control samples, ATP, buffers, etc. useful in the method. For example, preferred kits comprise: a) a 5′-phosphate-dependent nucleic acid exonuclease; b) a polynucleotide kinase (e.g., T4 polynucleotide kinase); and c) an RNA-dependent DNA polymerase. In some preferred kits in which the mRNA in the sample is known to be of sufficient quality for the intended purpose, meaning that approximately <90%, <75%, <50%, <40%, <30%, <20%, <15%, <10%, <5%, <4%, <3%, <2%, or <1% of the mRNA transcripts are degraded into fragments that lack a 5′-cap or a 5′-triphosphate group, the step comprising contacting the sample with the polynucleotide kinase in the presence of ATP and under conditions such that nucleic acid molecules having an hydroxyl group at their 5′-termini are phosphorylated can be omitted and a polynucleotide kinase is not needed in a preferred kit, or if included, its use can be omitted.

Similar methods and kits are provided that are useful for amplifying eukaryotic mRNA. For example, the present invention provides a method for amplifying eukaryotic mRNA, comprising the steps of a) providing a sample comprising RNA from a eukaryotic organism; and a 5′-phosphate-dependent nucleic acid exonuclease; b) contacting the sample with the 5′-phosphate-dependent nucleic acid exonuclease under conditions such that undesired nucleic acids in the sample are substantially digested but mRNA molecules in the sample having a 5′-triphosphate or a 5′-cap are not digested; and c) amplifying the mRNA. In preferred embodiments, the present invention provides a method for amplifying eukaryotic mRNA, comprising the steps of a) providing a sample comprising RNA from a eukaryotic organism; a polynucleotide kinase; and a 5′-phosphate-dependent nucleic acid exonuclease; b) contacting the sample with the polynucleotide kinase in the presence of ATP and under conditions such that nucleic acid molecules having an hydroxyl group at their 5′-termini are phosphorylated; c) contacting the sample with the 5′-phosphate-dependent nucleic acid exonuclease under conditions such that undesired nucleic acids in the sample are substantially digested but mRNA molecules in the sample having a 5′-triphosphate or a 5′-cap are not digested; and d) amplifying the mRNA. The present invention is not limited by the manner in which the mRNA is amplified. In some embodiments, the mRNA is amplified by a method comprising: i) contacting said mRNA molecules with an oligo(dT)-containing primer that anneals to a poly(A) tail of said mRNA and extending said primer with an RNA-dependent DNA polymerase under conditions whereby cDNA that is joined to an RNA polymerase promoter sequence is obtained (using any number of methods known in the art; methods are known for joining the promoter to either the 5′-end of the cDNA or the 3′-end of the cDNA, resulting in synthesis of amplified antisense or sense RNA, respectively, by subsequent in vitro transcription); ii) producing double stranded cDNA; and iii) contacting said double-stranded cDNA comprising said promoter sequence with an RNA polymerase that binds to said promoter and synthesizes RNA therefrom under conditions whereby RNA corresponding to mRNA in the biological sample is synthesized. Some other embodiments of the present invention use a single-stranded first-strand cDNA template that is functionally joined at either the 3′-end or the 5′-end to a single-stranded promoter that is recognized by an RNA polymerase, such as MiniV RNA polymerase, that can synthesize RNA in vitro therefrom; in these embodiments, the method does not comprise a step for synthesis of double-stranded cDNA. Still further, the invention also comprises methods for amplifying mRNA enriched or isolated using a 5′-phosphate-dependent exonuclease of the invention using methods, such as but not limited to, methods described in U.S. Pat. Appln. No. 2004/0180361 of Dahl et al.; PCT Patent Publication Nos. WO 02/16639; WO 00/56877; and AU 00/29742 of Takara Shuzo Company; and in U.S. Pat. No. 6,251,639 and U.S. Pat. Appln. Nos. 2001/0034048; 2003/0017591; 2003/0087251; and 2003/0186234 of Kurn (all incorporated herein by reference), even if said methods comprise synthesis of DNA products rather than RNA products corresponding to mRNA in the sample. Thus, another embodiment of the invention comprises a method for amplifying mRNA enriched or isolated using a 5′-phosphate-dependent exonuclease, the method comprising: i) contacting said mRNA molecules with a first primer that anneals to said mRNA and extending said first primer with an RNA-dependent DNA polymerase under conditions whereby first-strand cDNA is obtained; ii) contacting said first-strand cDNA with a second primer and extending said second primer with a strand-displacing DNA-dependent DNA polymerase under conditions whereby double-stranded cDNA that is joined to a ribonucleotide-containing tail sequence at the 3′-end of at least one cDNA strand is obtained; and iii) contacting said double-stranded cDNA with a ribonuclease H and a ribonucleotide-containing primer that anneals to the tail sequence and extending said ribonucleotide-containing primer with the strand-displacing DNA-dependent DNA polymerase under conditions whereby at least one strand of said cDNA is amplified. Kits for conducting such methods may have any one or more reagents (e.g., primers), control samples, buffers, etc. useful in the method. For example, preferred kits comprise: a) a 5′-phosphate-dependent nucleic acid exonuclease; b); c) an RNA-dependent DNA polymerase; and c) an RNA polymerase (e.g., T7, T3, or SP6 RNA polymerases). Other kits that use single-stranded templates and promoters use an RNA polymerase that synthesize RNA in vitro therefrom, such as MiniV RNA polymerase. Other preferred kits further comprise: a) a polynucleotide kinase, such as but not limited to T4 polynucleotide kinase, and ATP. Still other kits also include a ribonucleotide-containing primer, a ribonuclease H, and a strand-displacing DNA-dependent DNA polymerase, such as but not limited to phi29 DNA polymerase or rBST DNA polymerase large fragment (both available from EPICENTRE).

In some cases in which a sample cannot be replaced (e.g, a forensics sample from a crime scene), it may not be desirable to remove mRNA fragments that do not have a 5′-triphosphate or a 5′-cap prior to synthesis of cDNA and/or RNA amplification. Thus, some embodiments of the present invention comprise a method for preparing prokaryotic mRNA, eukaryotic mRNA or a mixture of both prokaryotic and eukaryotic mRNA that is degraded for synthesis of cDNA and/or RNA amplification, the method comprising: a) providing a sample comprising degraded RNA from a prokaryotic and/or eukaryotic organism; a poly(A) polymerase; and a 5′-phosphate-dependent nucleic acid exonuclease; b) contacting the sample with the 5′ exonuclease under conditions such that undesired nucleic acids in the sample are substantially digested but desired RNA molecules are not digested; and c) contacting the sample with a poly(A) polymerase in the presence of ATP and under conditions in which a 3′-poly(A) tail is added to the desired RNA molecules. The invention further comprises a method for preparing cDNA from said 3′-polyadenylated RNA, the method comprising: a) providing a sample comprising 3′-polyadenylated RNA; an RNA-dependent DNA polymerase; and an oligo(dT)-containing primer that anneals to said tail; and b) contacting the 3′-polyadenylated RNA with the oligo(dT)-containing primer and extending the primer with the RNA-dependent DNA polymerase under conditions whereby cDNA is obtained. Other embodiments further comprise methods for amplifying the polyadenylated RNA, the method comprising a) contacting the polyadenylated RNA with a primer that anneals to said tail and extending said primer with an RNA-dependent DNA polymerase under conditions whereby cDNA that is joined to an RNA polymerase promoter sequence is obtained (using any number of methods known in the art; methods are known for joining the promoter to either the 5′-end of the cDNA or the 3′-end of the cDNA, resulting in synthesis of amplified antisense or sense RNA, respectively, by subsequent in vitro transcription); b) producing double stranded cDNA; and c) contacting said double-stranded cDNA comprising said promoter sequence with an RNA polymerase that binds to said promoter and synthesizes RNA therefrom under conditions whereby amplified RNA corresponding to polyadenylated RNA in the sample is synthesized. Kits for conducting such methods may have any one or more reagents (e.g., primers, such as an oligo(dT) primer, or other oligonucleotides), control samples, buffers, etc. useful in the method. For example, preferred kits comprise: a) a 5′-phosphate-dependent nucleic acid exonuclease; and b) a poly(A) polymerase. Other preferred kits for preparing cDNA additionally comprise an RNA-dependent DNA polymerase. Still other preferred kits for methods comprising sense or antisense RNA amplification comprise, in addition to an RNA-dependent DNA polymerase, an RNA polymerase (e.g., without limitation, T7, T3, or SP6 RNA polymerase).

The present invention also provides a method for using a 5′-phosphate-dependent exonuclease of the invention to obtain 5′-ends of degraded mRNA and other primary transcripts that have a 5′-cap or a 5′-triphosphate group.

For example, one method that can be used to isolate the 5′-ends of degraded mRNA and other primary transcripts that have a 5′-cap or a 5′-triphosphate group comprises the steps of:

a) obtaining a sample of total RNA comprising degraded RNA;

b) treating the degraded RNA with a polynucleotide kinase (e.g., T4 polynucleotide kinase) under conditions that result in phosphorylation of 5′-hydroxyl groups of said degraded RNA;

c) treating the RNA from step (b) with a 5′-phosphate-dependent exonuclease of the invention under conditions that result in digestion of RNA having a 5′-phosphate group; and

d) isolating the RNA that is not digested in step (c).

Degraded RNA, such as but not limited to RNA degraded by RNase A-type nucleases, can have 5′-hydroxylated termini. The polynucleotide kinase phosphorylates the 5′-ends of degrade RNA molecules with a hydroxyl group, making these molecules subtrates which can be digested by the 5′-to-3′ exoribonuclease. The RNA obtained using this procedure will comprise 5′-end fragments of RNA that have a 5′-cap or a 5′-triphosphate. Without limiting the invention, if sufficient quantities of these fragments are available, the RNA fragments can be directly labeled [e.g., using ULS™ (Kreatech) or other chemical dye labeling compounds] and used for gene expression studies with arrays or microarrays of oligonucleotide sequences that are complementary to the 5′-ends of all known sequences or desired known sequences corresponding to mRNA transcripts for the organism from which the total RNA was obtained. If sufficient quantities of the RNA fragments are not available from this method (e.g., if the RNA fragments are obtained from only a single cell or a small number of cells), then the 5′-end fragments of RNA having a 5′-cap or a 5′-triphosphate obtained can be amplified by one of several methods. By way of example but not of limitation, the 5′-end fragments can be polyadenylated using poly(A) polymerase using methods known in the art, and then amplified using an Eberwine-type RNA amplification method (e.g., see Van Gelder et al., Proc. Nat. Acad. Sci. USA 87: 1663, 1990), which uses an oligo(dT) promoter primer and generates anti-sense RNA. A 1-round or 2-round TargetAmp™ aRNA or aminoallyl-aRNA Amplification Kit (EPICENTRE Technologies, Madison, Wis., USA) or another kit can also be used for this purpose. If the amplified and labeled aRNA is analyzed using a microarray, the oligos on the microarray must be complementary to the aRNA corresponding to the 5′-ends of the mRNA fragments rather than to the sense mRNA sequence. Alternatively, a sense mRNA amplification method, such as available in kits from BD-Clontech and from Genisphere can be use to amplify sense RNA corresponding to the 5′-ends of the mRNA fragments. A method comprising an oligonucleotide sequence tag template can be used to add a sequence tag to the 3′-end of a nucleic acid, including an RNA or DNA molecule, which can then be amplified following addition of a promoter, as described in U.S. Patent Application No. 2005/015333, which is incorporated herein by reference. After labeling, the amplified sense RNA can be annealed to a microarray of antisense oligos complementary to all known sequences or desired known sequences corresponding to the 5′-ends of mRNA transcripts for the organism from which the total RNA was obtained. Still further, the 5′-ends of the mRNA fragments can be amplified without polyadenylation by using a promoter primer with a random sequence for priming first-strand cDNA synthesis (e.g., see Ziman and Davis, WO 2002/044399). Still other methods for amplifying the mRNA 5′-end fragments are described herein which can be used as part of the present invention.

In other embodiments of the invention, a 5′-phosphate-dependent exonuclease is used to obtain 5′-end fragments from intact, undegraded mRNA or other primary transcripts having a 5′-cap or a 5′-triphosphate in a sample which contains other undesired nucleic acids. One way that the total RNA can be analyzed to determine that it is not significantly degraded is to determine the profile of large rRNA in the sample using an Agilent bioanalyzer and to verify that the scan of the rRNA indicates that the large rRNA is largely intact. If desired, small RNA, such as 5S rRNA and tRNA, can be substantially removed from the total RNA by precipitation of other RNA in the sample using LiCl and ethanol, leaving 5S rRNA and tRNA in solution, prior to performing additional steps to isolate 5′-end fragments of mRNA. Thus, one embodiment is a method for isolating the 5′-ends of mRNA and other primary transcripts that have a 5′-cap or a 5′-triphosphate group (i.e., the nucleic acid of interest) from a sample which also contains undesired nucleic acid (e.g., large rRNA in a sample of total RNA), wherein the nucleic acid of interest is not substantially degraded, the method comprising:

a) obtaining a sample of total RNA that contains mRNA;

b) optionally, obtaining an RNA fraction by that is reduced in 5S rRNA and tRNA LiCl and ethanol precipitation;

c) treating the RNA so as to bring about controlled degradation of the RNA into smaller fragments by physical or chemical means or by degradation with a nuclease;

d) treating the degraded RNA from step (c) with a polynucleotide kinase (e.g., T4 polynucleotide kinase) under conditions that result in phosphorylation of 5′-hydroxyl groups of said degraded RNA;

e) treating the RNA from step (d) with a 5′-to-3′ exoribonuclease under conditions that result in digestion of RNA having a 5′-phosphate group; and

f) isolating the RNA that is not digested in step (e).

This procedure will give a solution of fragments of 5′-capped or 5′-triphosphorylated mRNA of the desired size range. These mRNA 5′-end fragments can then be used for any desired application, including for the applications and methods described with respect to the 5′-end fragments of mRNA obtained from FFPE degraded RNA discussed herein. For example, but without limitation, the fragments can be polyadenylated using poly(A) polymerase and amplified using an RNA amplification method that yields labeled aRNA or sense RNA as described herein. In other embodiments of the invention, the 5′-end fragments of mRNA can be amplified and analyzed using rolling circle replication or rolling circle transcription as also described herein.

Some methods that can be used for treating the RNA so as to bring about controlled degradation of the RNA into smaller fragments include chemical treatments, such exposing the RNA to cations, including, but not limited to Mg²⁺ under elevated temperatures, and controlled degradation with a ribonuclease (after optimizing the enzyme, enzyme amount, time and other reactions conditions that yield RNA fragments in the desired size range). One RNase that can be used to fragment the RNA is RNase I, which cleaves the RNA after every base and which can be inactivated by heat after the treatment. Still another embodiment of a method of the invention for controlled degradation of RNA with a nuclease comprises treating the RNA with an RNase H in the presence of one or more DNA oligonucleotides complementary to specific sequences of the RNA of interest, under conditions wherein the DNA oligonucleotides can hybridize to the complementary mRNA and the RNase H is active.

Still another embodiment of the invention is a method for method for amplifying circularized cDNA, which cDNA is prepared from 5′-end fragments of 5′-capped or 5′-triphosphorylated RNA, by rolling circle replication. Thus, one embodiment of the invention is a method for amplifying the 5′-end fragments of mRNA and other primary transcripts, the method comprising the steps of:

a) providing a sample comprising 5′-end fragments of mRNA or other primary transcripts of a desired size range (e.g., without limitation, <100 bases);

b) contacting the 5′-end fragments with poly(A) polymerase under conditions that result in addition of a poly(A) tail of sufficient length to permit annealing of a primer for reverse transcription;

c) contacting the poly(A)-tailed 5′-end fragments with a 5′-phosphorylated primer that anneals to the poly(A) tail and an RNA-dependent DNA polymerase or reverse transcriptase under conditions that result in extension of the primer and synthesis of first-strand cDNA that is complementary to the poly(A)-tailed 5′-end fragments;

d) treating the complex between the first-strand cDNA and the poly(A)-tailed 5′-end fragments of mRNA from step (c) so as to remove the RNA from the first-strand cDNA;

e) contacting the first-strand cDNA from step (d) with a ligase that can ligate single-stranded DNA (ssDNA) in the absence of a ligation splint oligo under conditions that result in intramolecular ligation (i.e., circularization) of the linear first-strand cDNA so as to obtain circular first-strand cDNA; and

f) contacting the circular first-strand cDNA obtained from step (e) with a strand-displacing DNA polymerase (e.g., without limitation, phi29 DNA polymerase or rBst DNA polymerase large fragment) and a primer that anneals to the circular first-strand cDNA under conditions that result in synthesis of concatemeric second-strand cDNA that is complementary to the circular first-strand cDNA (e.g., by rolling circle replication).

The primer used to prime first-strand cDNA synthesis in step (c) can be an oligo(dT)ₙ or an oligo(dT)ₙX anchored primer, wherein X is a mixture of dAMP, dCMP and dGMP. In step (c) above, the primer has a 5′-phosphate. In other embodiments, the primer has a 5′-hydroxyl group and is phosphorylated using a polynucleotide kinase (e.g., T4 polynucleotide kinase) under kinasing conditions after step (d) and prior to step (e). An RNA-dependent DNA polymerase or reverse transcriptase that does not have RNase H activity, such as but not limited to, an enzyme is selected from among an RNase H-minus mutant of MMLV or AMV reverse transcriptase (e.g., SuperScript™ I, II, or III from Invitrogen) can be used and is preferred in step (c), but an RNA-dependent DNA polymerase that has RNase H activity can be used in some embodiments. Any ligase that ligates ssDNA in the absence of a ligation splint oligo can be used, such as but not limited to, a ligase selected from the group consisting of a ligase derived from phage TS2126 that infect Thermus scotoductus, CircLigase™ ssDNA ligase (EPICENTRE), and ThermoPhage™ ssDNA Ligase (PROKARIA, Reykjavik, Iceland). In some embodiments, the primer used for rolling circle replication in step (f) is an oligo(dA) primer that anneals to the oligo(dT) sequence from the primer that was used to prime synthesis of first-strand cDNA. In other embodiments, a multiplicity of primers is used for rolling circle replication. For example, in one embodiment, a solution comprising all possible combinations of short primers such as hexamers (i.e., the hexamer is synthesized to contain any of the four canonical nucleotide bases at each of the six nucleotide positions) is used. In some aspects of this embodiment, the concatemeric rolling circle replication product is labeled by incorporation of a labeled nucleotide, but any other suitable method known in the art can also be used to detect the rolling circle replication product. In another embodiment in which a multiplicity of primers is used for rolling circle replication, the primers comprise oligonucleotides that have a sequence that is the same as at least one sequence within a certain defined distance of the 5′-end of the known mRNA transcripts for the organism for which transcription is analyzed. The defined distance from the 5′-end of the mRNA transcripts within which the complementary sequence for the priming oligos is chosen is similar to the average length of the 5′-end fragments provided in step (a). In a preferred aspect of this embodiment, the multiplicity of primers having the same sequence as at least one sequence within a certain defined distance of the 5′-end of the known mRNA transcripts for the organism for which transcription is analyzed are attached to a surface (e.g., in an array or microarray) so that their 3′-ends have an hydroxyl group and the sequences are at sufficient distance from the surface so that the sequences can prime rolling circle replication by primer extension of the tethered primer using a circular first-strand cDNA from step (e), to which the primer is complementary, as a template. In this way, the primer that is attached to the surface is primer extended by rolling circle replication using a strand-displacing DNA polymerase under polymerization conditions. In some aspects of this embodiment, the concatemeric rolling circle replication product is labeled by incorporation of a labeled nucleotide, but any other suitable method known in the art can also be used to detect the rolling circle replication product.

Yet another method that can be used to amplify 5′-end fragments of mRNA and other primary transcripts comprises the steps of:

a) providing a sample comprising 5′-end fragments of mRNA and other primary transcripts of a desired size range (e.g., without limitation, ≦100 bases);

b) contacting the 5′-end fragments with a 5′-phosphorylated random hexamer primer and an RNA-dependent DNA polymerase or reverse transcriptase under conditions that result in extension of the primer and synthesis of first-strand cDNA that is complementary to the 5′-end fragments;

c) treating the complex between the first-strand cDNA and the 5′-end fragments of mRNA from step (b) so as to remove the RNA from the first-strand cDNA;

d) contacting the first-strand cDNA from step (c) with a ligase that can ligate single-stranded DNA (ssDNA) in the absence of a ligation splint oligo under conditions that result in intramolecular ligation (i.e., circularization) of the linear first-strand cDNA so as to obtain circular first-strand cDNA; and

e) contacting the circular first-strand cDNA obtained from step (d) with a strand-displacing DNA polymerase (e.g., without limitation, phi29 DNA polymerase or rBst DNA polymerase large fragment) and a primer that anneals to the circular first-strand cDNA under conditions that result in synthesis of concatemeric second-strand cDNA that is complementary to the circular first-strand cDNA (e.g., by rolling circle replication).

In preferred embodiments this method, the RNA-dependent DNA polymerase or reverse transcriptase is an enzyme that does not have RNase H activity. By way of example, but not of limitation, the enzyme is selected from among an RNase H-minus mutant of MMLV or AMV reverse transcriptase (e.g., SuperScript™ I, II, or III from Invitrogen). A ligase that ligates ssDNA in the absence of a ligation splint oligo includes, but is not limited to a ligase selected from the group consisting of a ligase derived from phage TS2126 that infect Thermus scotoductus, CircLigase™ ssDNA ligase (EPICENTRE), and ThermoPhage™ ssDNA Ligase (PROKARIA, Reykjavik, Iceland). In some embodiments, a multiplicity of primers is used for step (e). For example, in one embodiment, a solution comprising all possible hexamer primers (i.e., the hexamer is synthesized to contain any of the four canonical nucleotide bases at each of the six positions). In some aspects of this embodiment, the concatemeric rolling circle replication product is labeled by incorporation of a labeled nucleotide, but any other suitable method known in the art can also be used to detect the rolling circle replication product. In other embodiments in which a multiplicity of primers is used for rolling circle replication, the primers comprise oligonucleotides that have a sequence that is the same as at least one sequence within a certain defined distance of the 5′-end of the known mRNA transcripts for the organism for which transcription is analyzed. The defined distance from the 5′-ends of the mRNA transcripts is chosen to be similar to the average length of the 5′-end fragments provided in step (a). In a preferred aspect of this embodiment, the multiplicity of primers having the same sequence as at least one sequence within a certain defined distance of the 5′-end of the known mRNA transcripts for the organism for which transcription is analyzed are attached to a surface (e.g., in an array or microarray) so that their 3′-ends have an hydroxyl group and the sequences are at sufficient distance from the surface so that the sequences can prime rolling circle replication by primer extension of the tethered primer using a circular first-strand cDNA from step (d), to which the primer is complementary, as a template. In this way, the primer that is attached to the surface is primer extended by rolling circle replication using a strand-displacing DNA polymerase under polymerization conditions. In some aspects of this embodiment, the concatemeric rolling circle replication product is labeled by incorporation of a labeled nucleotide, but any other suitable method known in the art can also be used to detect the rolling circle replication product.

Another method for amplifying circularized cDNA prepared from 5′-end fragments of 5′-capped or 5′-triphosphorylated RNA is by rolling circle transcription. Thus, one method that can be used to amplify the 5′-end fragments of mRNA and other primary transcripts comprises the steps of:

a) providing a sample comprising 5′-end fragments of mRNA and other primary transcripts of a desired size range (e.g., without limitation, ≦100 bases);

b) contacting the 5′-end fragments with poly(A) polymerase under conditions that result in addition of a poly(A) tail of sufficient length to permit annealing of a reverse transcription primer;

c) contacting the poly(A)-tailed 5′-end fragments with a 5′-phosphorylated primer that anneals to the poly(A) tail and an RNA-dependent DNA polymerase or reverse transcriptase under conditions that result in extension of the primer and synthesis of first-strand cDNA that is complementary to the poly(A)-tailed 5′-end fragments;

d) treating the complex between the first-strand cDNA and the poly(A)-tailed 5′-end fragments of mRNA from step (c) so as to remove the RNA from the first-strand cDNA;

e) contacting the first-strand cDNA from step (d) with a ligase that can ligate single-stranded DNA (ssDNA) in the absence of a ligation splint oligo under conditions that result in intramolecular ligation (i.e., circularization) of the linear first-strand cDNA so as to obtain circular first-strand cDNA; and

f) contacting the circular first-strand cDNA obtained from step (e) with an RNA polymerase under conditions that result in synthesis of concatemeric RNA complementary to the first-strand cDNA by rolling circle transcription.

An oligo(dT)ₙ or an oligo(dT)ₙX anchored primer, wherein X=a mixture of dAMP, dCMP and dGMP, can be used to prime first-strand cDNA synthesis in step (c). In step (c) above, the primer has a 5′-phosphate. In other embodiments, the primer has a 5′-hydroxyl group and is phosphorylated using a polynucleotide kinase (e.g., T4 polynucleotide kinase) under kinasing conditions after step (d) and prior to step (e). RNA-dependent DNA polymerases or reverse transcriptases that can be used include, but are not limited to enzymes that lack RNase H activity. By way of example, but not of limitation, the enzyme can be selected from among an RNase H-minus mutant of MMLV or AMV reverse transcriptase (e.g., SuperScript™ I, II, or III from Invitrogen). However, a reverse transcriptase with RNase H activity can also be used. Ligases that can be used to ligate ssDNA include, but are not limited to, ligases selected from the group consisting of a ligase derived from phage TS2126 that infect Thermus scotoductus, CircLigase™ ssDNA ligase (EPICENTRE), and ThermoPhage™ ssDNA Ligase (PROKARIA, Reykjavik, Iceland). The RNA polymerase can be any RNA polymerase that transcribes a single-stranded circular DNA in the absence of a primer and in the absence of a promoter sequence, although a promoter sequence can be present in some embodiments of the invention. RNA polymerases that can be used include, but are not limited to, E. coli RNA polymerase (including core enzyme); a T7-type RNA polymerase, including T7 RNAP, T3 RNAP, and SP6 RNAP; or phage N4 mini-vRNAP.

Still another method that can be used to amplify the 5′-end fragments of mRNA and other primary transcripts comprises the steps of:

a) providing a sample comprising 5′-end fragments of mRNA and other primary transcripts of a desired size range (e.g., without limitation, ≦100 bases);

b) contacting the 5′-end fragments with a 5′-phosphorylated random hexamer primer and an RNA-dependent DNA polymerase or reverse transcriptase under conditions that result in extension of the primer and synthesis of first-strand cDNA that is complementary to the 5′-end fragments;

c) treating the complex between the first-strand cDNA and the 5′-end fragments of mRNA from step (b) so as to remove the RNA from the first-strand cDNA;

d) contacting the first-strand cDNA from step (c) with a ligase that can ligate single-stranded DNA (ssDNA) in the absence of a ligation splint oligo under conditions that result in intramolecular ligation (i.e., circularization) of the linear first-strand cDNA so as to obtain circular first-strand cDNA; and

e) contacting the circular first-strand cDNA obtained from step (d) with an RNA polymerase under conditions that result in synthesis of concatemeric RNA complementary to the first-strand cDNA by rolling circle transcription.

RNA-dependent DNA polymerases or reverse transcriptases that can be used include, but are not limited to enzymes that lack RNase H activity. By way of example, but not of limitation, the enzyme can be selected from among an RNase H-minus mutant of MMLV or AMV reverse transcriptase (e.g., SuperScript™ I, II, or III from Invitrogen). However, a reverse transcriptase with RNase H activity can also be used. Ligases that can be used to ligate ssDNA include, but are not limited to, ligases selected from the group consisting of a ligase derived from phage TS2126 that infect Thermus scotoductus, CircLigase™ ssDNA ligase (EPICENTRE), and ThermoPhage™ ssDNA Ligase (PROKARIA, Reykjavik, Iceland). The RNA polymerase can be any RNA polymerase that transcribes a single-stranded circular DNA in the absence of a primer and in the absence of a promoter sequence, although a promoter sequence can be present in some embodiments of the invention. RNA polymerases that can be used include, but are not limited to, E. coli RNA polymerase (including core enzyme); a T7-type RNA polymerase, including T7 RNAP, T3 RNAP, and SP6 RNAP; or phage N4 mini-vRNAP.

The present invention also comprises embodiments comprising amplification of circular first-strand cDNA by both rolling circle replication and rolling circle transcription using any combination of the methods described herein.

The present invention further comprises methods methods, compositions, and kits for amplification of linear first-strand cDNA prepared from 5′-capped or 5′-triphosphorylated mRNA using a random hexamer primer and a strand-displacing DNA polymerase. Thus, one method that can be used to amplify the 5′-end fragments of mRNA and other primary transcripts comprises the steps of:

a) providing a sample comprising 5′-end fragments of mRNA and other primary transcripts of a desired size range (e.g., without limitation, approximately 100-500 bases);

b) contacting the 5′-end fragments with poly(A) polymerase under conditions that result in addition of a poly(A) tail of sufficient length to permit annealing of a reverse transcription primer;

c) contacting the poly(A)-tailed 5′-end fragments with a primer that anneals to the poly(A) tail and an RNA-dependent DNA polymerase or reverse transcriptase under conditions that result in extension of the primer and synthesis of first-strand cDNA that is complementary to the poly(A)-tailed 5′-end fragments;

d) treating the complex between the first-strand cDNA and the poly(A)-tailed 5′-end fragments of mRNA from step (c) so as to remove the RNA from the first-strand cDNA; and

e) contacting the first-strand cDNA obtained from step (d) with a strand-displacing DNA polymerase (e.g., phi29 DNA polymerase) and a random hexamer primer that anneals to the first-strand cDNA under conditions that result in amplification of the cDNA.

An oligo(dT)ₙ or an oligo(dT)ₙX anchored primer, wherein X=a mixture of dAMP, dCMP and dGMP, can be used to prime first-strand cDNA synthesis in step 4.A. (c). In preferred embodiments of step 4.A. (c), the RNA-dependent DNA polymerase or reverse transcriptase is an enzyme that does not have RNase H activity. By way of example, but not of limitation, the enzyme is selected from among an RNase H-minus mutant of MMLV or AMV reverse transcriptase (e.g., SuperScript™ I, II, or III from Invitrogen). However, reverse transcriptases with RNase H activity can be used in some embodiments. In some embodiments of step 4.A (e), phi29 DNA polymerase is used as the strand-displacing DNA polymerase and the random hexamer primer used comprises either a random hexamer DNA primer having alpha-thiophosphate internucleoside linkages, which are resistant to many exonucleases, or a random hexamer RNA primer.

Still another embodiment of the invention comprises a method for amplifying the 5′-end fragments of mRNA and other primary transcripts, the method comprising the steps of:

a) providing a sample comprising 5′-end fragments of mRNA and other primary transcripts of a desired size range (e.g., without limitation, approximately 100-500 bases);

b) contacting the 5′-end fragments with a random primer that anneals to 5′-end fragments of mRNA and other primary transcripts and an RNA-dependent DNA polymerase or reverse transcriptase under conditions that result in extension of the primer and synthesis of first-strand cDNA that is complementary to the 5′-end fragments;

c) treating the complex between the first-strand cDNA and the 5′-end fragments of mRNA from step (b) so as to remove the RNA from the first-strand cDNA; and

d) contacting the first-strand cDNA obtained from step (c) with a strand-displacing DNA polymerase (e.g., phi29 DNA polymerase) and a random hexamer primer that anneals to the first-strand cDNA under conditions that result in amplification of the cDNA.

The random primer used in step 4.B. (b) can comprise a random hexamer DNA primer having alpha-thiophosphate internucleoside linkages. In preferred embodiments of step 4.A. (b), the RNA-dependent DNA polymerase or reverse transcriptase is an enzyme that does not have RNase H activity. By way of example, but not of limitation, the enzyme is selected from among an RNase H-minus mutant of MMLV or AMV reverse transcriptase (e.g., SuperScript™ I, II, or III from Invitrogen). However, reverse transcriptases with RNase H activity can be used in some embodiments. The primer in step 4.B. (d) can comprise either a random hexamer DNA primer having alpha-thiophosphate internucleoside linkages or a random hexamer RNA primer. One DNA polymerase that can be used in this step is phi29 DNA polymerase.

The present invention further provides kits and methods for selectively modifying nucleic acids that are not substrates for a 5′ exonuclease of the present invention so that said nucleic acids will be substrates for said 5′ exonuclease and thereby, can be removed from desired nucleic acid molecules. For example, but without limitation, an RNA molecule that is not a substrate for a 5′-phosphate-dependent exoribonuclease because it has a triphosphate group or an hydroxyl group on its 5′-terminus can be hybridized to a DNA oligo that anneals near to its 5′-end and the RNA:oligo complex can be contacted with a ribonuclease H, such as but not limited to E. coli RNase H or a Thermus RNase H (e.g., Hybridase RNase H from EPICENTRE). The RNase H digests the RNA within the portion that is annealed to the oligo, yielding an RNA molecule that has a 5′-phosphorylated RNA that is a substrate for the 5′-phosphate-dependent exoribonuclease that can be substantially digested in the presence of desired nucleic acids. Thus, in some preferred embodiments, the present invention provides a method for enriching for a desired RNA in the presence of an undesired RNA comprising the steps of: a) providing a 5′-phosphate-dependent nucleic acid exoribonuclease; a sample comprising RNA, said sample containing desired RNA and an undesired RNA that is not a substrate for the exoribonuclease; a DNA oligo that is complementary to a sequence near the 5′-terminus of the undesired RNA; and an RNase H; b) contacting the sample with the DNA oligo and the RNase H under conditions under which the oligo anneals to the undesired RNA and the RNase H is active; c) treating the sample with the exoribonuclease under conditions such that the undesired RNA is substantially digested and the desired RNA is not digested; and d) recovering the desired RNA (e.g., for additional analysis or use). In some embodiments, the 5′-phosphate-dependent nucleic acid exoribonuclease used in the method is provided in a kit that contains the DNA oligo, RNase H, buffers, instructions, and optionally, appropriate control samples, and the like.

The present invention is not limited with respect to kits and methods for modifying nucleic acids to be either desired or undesired nucleic acids that are, respectively, either resistant to digestion or susceptible to digestion by a 5′ exonuclease of the present invention. Any appropriate modification method can be used to achieve a particular purpose in a method of the invention. For example, but without limitation, a 5′-exonuclease-resistant nucleic acid with a 5′-hydroxyl can be modified to a 5′-phosphorylated nucleic acid that is a substrate for digestion by treatment with a polynucleotide kinase, such as T4 polynucleotide kinase, and ATP under kinase reaction conditions; or a 5′-phosphorylated nucleic acid that is a substrate for digestion by a 5′-exonuclease can be dephosphorylated to a 5′-exonuclease-resistant nucleic acid with a 5′-hydroxyl by treatment with a DNA phosphatase, such as, but not limited to APex Alkaline Phosphatase (EPICENTRE) or shrimp alkaline phosphatase; or a 5′-exonuclease-resistant 5′-triphosphorylated or 5′-capped mRNA can be modified to a 5′-exonuclease substrate that has a 5′-phosphate by treatment with a pyrophosphatase, such as but not limited to, tobacco acid pyrophosphatase. Those with skill in the art will know or know how to identify other methods for modifying undesired nucleic acids so that they will be substrates for a 5′ exonuclease of the invention or for modifying desired nucleic acids so they will not be substrates for the 5′ exonuclease, all of which modification methods are included within the scope of the present invention.

The present invention further provides methods and kits for removing undesired 5′-phosphorylated oligonucleotides from nucleic acid reactions (e.g., without limitation, aRNA and sense RNA amplification reactions), comprising the steps of: a) providing a sample that contains desired nucleic acid molecules that have undergone or are undergoing enzymatic manipulation and undesired 5′-phosphorylated oligonucleotides that are involved in said manipulation of said desired nucleic acid molecules; and b) exposing the sample to a 5′-phosphate-dependent nucleic acid exonuclease under conditions such that the undesired 5′-phosphorylated oligonucleotides are substantially digested and the desired nucleic acid molecules are not digested. The method finds use in a wide variety of applications. By way of example, but not of limitation, U.S. Patent Appln. Nos. 2004/0171041 and 2004/0197802, which are incorporated herein by reference in their entireties, disclose methods for which this aspect of the present invention can be used. For example, the present method can be used for methods disclosed therein that use a splice template oligo (or template switch oligo), such as a promoter splice template oligo to synthesize first-strand cDNA comprising a target sequence in order to reduce subsequent background transcription due to the presence of the splice template oligo if it is not removed. The splice template oligo can have a 5′-phosphate group or it can be phosphorylated during the method using a polynucleotide kinase. The present invention is not limited to removal of splice template oligos. The invention also includes removal of other oligos that are undesired nucleic acids. (e.g., removal of linear promoter primers from circular first-strand cDNA synthesized by primer extension of the promoter primer and subsequent ligation using a ligase in the presence of a ligation splint oligo or using a ligase that ligates ssDNA in the absence of a ligation splint oligo (e.g., CircLigase ssDNA Ligase (EPICENTRE Biotechnologies)), and the like, and from RNA amplification reactions). In some embodiments, the undesired 5′-phosphorylated oligonucleotide includes, but is not limited to, primers, ligation splint oligos, etc. In some embodiments, the enzymatic manipulation comprises a ligation reaction, a nucleic acid synthesis reaction (e.g., in vitro transcription reaction, cDNA synthesis reaction, Eberwine-type aRNA amplification reaction, sense RNA amplification reaction, etc.). Kits for conducting such methods comprise a 5′-phosphate-dependent exonuclease and may have any one or more other reagents (e.g., primers), control samples (e.g., 5′-phosphorylated oligonucleotides), buffers, etc. useful in the method.

The compositions and methods of the present invention provided herein can be used in diagnostic methods. For example, the present invention provides a method for detecting the presence of a target nucleic acid in a sample, comprising the steps of: a) providing a sample comprising a mixture of nucleic acids, said mixture of nucleic acids suspected of containing the target nucleic acid; b) exposing the sample to a 5′-phosphate-dependent nucleic acid exonuclease; and c) detecting the presence of undigested nucleic acid molecules that are not substrates for the exonuclease or detecting the products and/or process of digestion of nucleic acid molecules that are substrates for the exonuclease in order to detect the presence of the target nucleic acid in the sample. The method is not limited by the nature of the target nucleic acid. In some embodiments, the target nucleic acid, if present in the sample, is modified as discussed herein above to make it susceptible or resistant to cleavage by the 5′-phosphate-dependent nucleic acid exonuclease in a target-specific manner. The target nucleic acid may also be differentiated from non-target sequence by its different behavior in response to particular modification treatments (e.g., susceptibility to RNase H digestion in the presence of a DNA oligo or restriction enzyme digestion due to the presence of a particular sequence). The present invention also provides kits, mixtures, and compositions for carrying out such methods. For example, the present invention provides a kit for isolating a nucleic acid molecule of interest in a mixture of nucleic acids comprising one or more of: a) a 5′-phosphate-dependent nucleic acid exonuclease; b) a positive control sample comprising nucleic acid having a 5′ phosphate (e.g., rRNA); and c) a negative control sample comprising nucleic acid that is resistant to degradation by the 5′-phosphate-dependent nucleic acid exonuclease (e.g., mRNA).

The present invention further provides a method for isolating 5′-capped or 5′-triphosphorylated or 5′-hydroxylated small RNA molecules in samples containing eukaryotic RNA (e.g., small nuclear RNA (snRNA) or pre-microRNA (pre-mRNA) to study the their functions in gene processing and control of gene expression, etc. in health and disease) or 5′-capped or 5′-triphosphorylated transcripts of other small RNA molecules, such as but not limited to, primary, the method comprising the steps of: a) providing a sample comprising nuclear RNA from a eukaryotic organism (e.g., obtained from isolated nuclei or from total RNA preparations using methods known in the art), said sample containing desired small RNA having a 5′-cap or 5′-triphosphate or 5′-hydroxyl group and at least one undesired nucleic acid; and a 5′-to-3′ exoribonuclease; b) treating the sample with the 5′- to-3′ exoribonuclease under conditions such that said undesired nucleic acid is substantially digested and the desired 5′-capped or 5′-triphosphorylated or 5′-hydroxylated small RNA is not digested; and c) recovering the capped or triphosphorylated or hydroxylated small RNA. In some embodiments, the 5′-phosphate-dependent nucleic acid exonuclease used in the method is provided in a kit for isolation of capped or triphosphorylated or hydroxylated small RNA, with buffers, instructions, and optionally, with appropriate control samples, and the like. Pre-mRNA, which has a 5′-triphosphate, is further processed in the cell to micro RNA (mRNA) having a 5′-monophosphate, which is implicated in control of gene expression of many. In some embodiments of methods of the invention, a 5′-group is modified in order to achieve a purpose. For example, without limitation, in one embodiment of the method, small RNA with a 5′-phosphate group can be removed using a nucleic acid phosphatase, such as APex Phosphatase (EPICENTRE), to form a 5′-hydoxyl group, which is resistant to digestion with the 5′-to-3 exoribonuclease, thereby permitting enrichment for the small RNA (such as mRNA). In another embodiment, the small RNA with a 5′-triphosphate or 5′-cap can be treated with tobacco acid pyrophosphatase (EPICENTRE) to form a 5′-phosphate group, thereby permitting digestion of the small RNA, such as pre-mRNA, with the 5′-to-3 exoribonuclease.

The present invention further comprises methods to prepare cDNA from said isolated capped or triphosphorylated small RNA, the method comprising a) providing a sample comprising capped and/or triphosphorylated small RNA; a poly(A) polymerase; and an RNA-dependent DNA polymerase; b) contacting the sample with the poly(A) polymerase in the presence of ATP and under conditions in which a 3′-poly(A) tail is added to the desired small RNA molecules; and c) contacting the 3′-polyadenylated RNA with the oligo(dT)-containing primer and extending the primer with the RNA-dependent DNA polymerase under conditions whereby cDNA is obtained. Other embodiments comprise methods for amplifying the polyadenylated small RNA, the methods comprising a) contacting the polyadenylated small RNA with a primer that anneals to said tail and extending said primer with an RNA-dependent DNA polymerase under conditions whereby cDNA that is joined to an RNA polymerase promoter sequence is obtained (using any number of methods known in the art; methods are known for joining the promoter to either the 5′-end of the cDNA or the 3′-end of the cDNA, resulting in synthesis of amplified antisense or sense RNA, respectively, by subsequent in vitro transcription); b) producing double stranded cDNA; and c) contacting said double-stranded cDNA comprising said promoter sequence with an RNA polymerase that binds to said promoter and synthesizes RNA therefrom under conditions whereby amplified RNA corresponding to polyadenylated RNA in the sample is synthesized. Kits for conducting such methods may have any one or more reagents (e.g., primers), control samples, buffers, etc. useful in the method. For example, preferred kits comprise: a) a 5′-phosphate-dependent nucleic acid exonuclease; b) a poly(A) polymerase; c) an RNA-dependent DNA polymerase; and, for methods comprising RNA amplification, d) an RNA polymerase (e.g., T7, T3, and SP6 RNA polymerases).

One preferred embodiment of the invention is a kit for enrichment of mRNA from a sample comprising mRNA and at least one undesired nucleic acid, the kit comprising a 5′-to-3′ exoribonuclease that has an activity in digesting an RNA having a 5′-triphosphate or a 5′-cap that is less than 5% of its activity in digesting said RNA having a 5′-monophosphate.

Another preferred embodiment is a kit for enrichment of mRNA from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; and b) a negative control comprising an RNA having a 5′-triphosphate or a 5′-cap.

Another preferred embodiment is a kit for enrichment of mRNA from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; and b) a positive control comprising an RNA having a 5′-monophosphate.

Still another preferred embodiment is a kit for enrichment of mRNA from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a negative control comprising an RNA having a 5′-triphosphate or a 5′-cap; and c) a positive control comprising an RNA having a 5′-monophosphate.

Another preferred embodiment of the invention is a kit for isolation of mRNA from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; and b) a solution of LiCl for precipitation of low molecular RNA.

Another preferred embodiment is a kit for isolation of mRNA from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a solution of LiCl for precipitation of low molecular RNA; and c) a negative control comprising an RNA having a 5′-triphosphate or a 5′-cap.

Another preferred embodiment is a kit for isolation of mRNA from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a solution of LiCl for precipitation of low molecular RNA; and c) a positive control comprising an RNA having a 5′-monophosphate.

Still another preferred embodiment is a kit for isolation of mRNA from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a solution of LiCl for precipitation of low molecular RNA; c) a negative control comprising an RNA having a 5′-triphosphate or a 5′-cap; and d) a positive control comprising an RNA having a 5′-monophosphate.

The present invention further comprises a kit for enrichment of mRNA having a 5′-triphosphate or a 5′-cap from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; and b) a polynucleotide kinase.

Another embodiment of the invention is a kit for isolation of mRNA having a 5′-triphosphate or a 5′-cap from a sample comprising mRNA and at least one undesired nucleic acid, the kit comprising: a) a 5′-to-3′ exoribonuclease, wherein the an activity in digesting an RNA having a 5′-triphosphate or a 5′-cap is less than 5% of its activity in digesting said RNA having a 5′-monophosphate; b) a polynucleotide kinase; and c) a solution of LiCl for precipitation of low molecular RNA.

The present invention further comprises a kit for synthesis of first-strand cDNA complementary to mRNA from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) at least one primer that anneals to said mRNA in said sample; and c) an RNA-dependent DNA polymerase. One kit according to this embodiment is a kit for synthesis of first-strand cDNA complementary to mRNA from a prokaryotic organism. Another kit according to this embodiment is a kit for synthesis of first-strand cDNA complementary to mRNA from a eukaryotic organism. Still another kit according to this embodiment is a kit for synthesis of first-strand cDNA complementary to mRNA from both a prokaryotic organism and a eukaryotic organism.

Still further, another preferred embodiment of the invention is a kit for synthesis of first-strand cDNA complementary to mRNA from a prokaryotic organism, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a poly(A) polymerase; c) an oligo(dT)-containing primer that is capable of annealing to a poly(A) tail; and d) an RNA-dependent DNA polymerase. Another kit according to this embodiment is a kit for synthesis of first-strand cDNA complementary to mRNA from both a prokaryotic organism and a eukaryotic organism in a sample comprising mRNA from both the prokaryotic and the eukaryotic organisms.

Still another preferred embodiment of the invention is a kit for synthesis of first-strand cDNA complementary to mRNA from a prokaryotic organism, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a poly(A) polymerase; c) a polynucleotide kinase; d) an oligo(dT)-containing primer that is capable of annealing to a poly(A) tail; and e) an RNA-dependent DNA polymerase. Another kit according to this embodiment is a kit for synthesis of first-strand cDNA complementary both to mRNA from a prokaryotic organism and to full-length mRNA from a eukaryotic organism in a sample comprising mRNA from both the prokaryotic and the eukaryotic organisms.

Yet another preferred embodiment of the present invention is a kit for synthesis of first-strand cDNA complementary to full-length mRNA from a eukaryotic organism, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a polynucleotide kinase; c) an oligo(dT)-containing primer that is capable of annealing to a poly(A) tail; and d) an RNA-dependent DNA polymerase.

Still further, the invention also comprises a kit for RNA amplification of mRNA from a sample comprising mRNA and at least one undesired nucleic acid, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) at least one primer that anneals to said mRNA in said sample; c) an oligonucleotide that encodes an RNA polymerase promoter; d) an RNA-dependent DNA polymerase; and e) an RNA polymerase that binds to the RNA polymerase promoter encoded by the oligonucleotide. One kit according to this embodiment is a kit for RNA amplification of mRNA from a prokaryotic organism. Another kit according to this embodiment is a kit for RNA amplification of mRNA from a eukaryotic organism. Still another kit according to this embodiment is a kit for RNA amplification of mRNA from both a prokaryotic organism and a eukaryotic organism.

One preferred embodiment of the invention is a kit for RNA amplification of mRNA from a prokaryotic organism, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a poly(A) polymerase; c) an oligo(dT)-containing primer that is capable of annealing to a poly(A) tail; d) an oligonucleotide that encodes an RNA polymerase promoter; e) an RNA-dependent DNA polymerase; and f) an RNA polymerase that binds to the RNA polymerase promoter encoded by the oligonucleotide. One kit according to this embodiment of the invention is a kit for RNA amplification of mRNA from both a prokaryotic organism and a eukaryotic organism in a sample comprising mRNA from both the prokaryotic and the eukaryotic organisms.

Still another preferred embodiment of the invention is a kit for RNA amplification of mRNA from a prokaryotic organism, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a poly(A) polymerase; c) a polynucleotide kinase; d) an oligo(dT)-containing primer that is capable of annealing to a poly(A) tail; e) an oligonucleotide that encodes an RNA polymerase promoter; e) an RNA-dependent DNA polymerase; and f) an RNA polymerase that binds to the RNA polymerase promoter encoded by the oligonucleotide. One kit according to this embodiment is a kit for RNA amplification of both mRNA from a prokaryotic organism and full-length mRNA from a eukaryotic organism in a sample comprising mRNA from both the prokaryotic and the eukaryotic organisms.

The present invention further comprises a kit for RNA amplification of mRNA from a eukaryotic organism, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) an oligo(dT)-containing primer that is capable of annealing to a poly(A) tail; c) an oligonucleotide that encodes an RNA polymerase promoter; d) an RNA-dependent DNA polymerase; and e) an RNA polymerase that binds to the RNA polymerase promoter encoded by the oligonucleotide.

A preferred embodiment of the invention comprises a kit for RNA amplification of full-length mRNA from a eukaryotic organism, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; b) a polynucleotide kinase; c) an oligo(dT)-containing primer that is capable of annealing to a poly(A) tail; d) an oligonucleotide that encodes an RNA polymerase promoter; e) an RNA-dependent DNA polymerase; and f) an RNA polymerase that binds to the RNA polymerase promoter encoded by the oligonucleotide.

The present invention also comprises a kit for removing ribosomal RNA from a sample, the kit comprising: a) a 5′-to-3′ exoribonuclease, wherein the activity in digesting an RNA having a 5′-triphosphate or a 5′-cap is less than 5% of its activity in digesting said RNA having a 5′-monophosphate; and b) a positive control comprising rRNA selected from among a) 16S and 23S prokaryotic rRNA; and b) 18S and 26S or 28S eukaryotic rRNA. A preferred kit of this embodiment comprises a kit for removing ribosomal RNA >300 nucleotides from a sample. Other kits according to these embodiments comprise kits that additionally comprise a negative control comprising an RNA having a 5′-triphosphate, a 5′-cap, or a 5′-hydroxyl.

One preferred embodiment of the invention is a kit for enriching for mRNA having a 5′-triphosphate or a 5′-cap in a biological sample comprising prokaryotic mRNA, eukaryotic mRNA or both prokaryotic and eukaryotic mRNA and at least one undesired nucleic acid, the kit comprising: (i) a solution of purified and stabilized 5′-to-3′ exoribonuclease; and (ii) a concentrated solution of a reaction buffer for providing a 1× reaction buffer in which the exoribonuclease is active. One suitable 1× concentration of a reaction buffer that can be used for the invention comprises: 50 mM Tris-HCl (pH 8.0), 2.0 mM MgCl₂, and 100 mM NaCl.

In another embodiment, a kit for enriching for mRNA additionally comprises one or more components selected from the group consisting of: (i) a negative control comprising an RNA having a 5′-triphosphate or a 5′-cap; (ii) a positive control comprising an RNA having a 5′-monophosphate; (iii) a solution of LiCl; (iv) a polynucleotide kinase; and (v) a poly(A) polymerase.

In still another embodiment, the kit additionally comprises an RNA-dependent DNA polymerase (reverse transcriptase).

In still another embodiment, the kit additionally comprises one or more components selected from the group consisting of: (i) a poly(A) polymerase; (ii) an oligo(dT)-containing primer; and (iii) an RNA-dependent DNA polymerase.

In yet another embodiment, the kit additionally comprises one or more components selected from the group consisting of: (i) a polynucleotide kinase; (ii) an oligo(dT)-containing primer; and (iii) an RNA-dependent DNA polymerase for obtaining cDNA complementary to full-length mRNA from a eukaryotic organism.

In yet another embodiment, the kit additionally comprises one or more primers selected from the group consisting of (i) an oligo(dT)-containing primer; and (ii) a primer that is complementary to a specific nucleic acid sequence. In some aspects of these embodiments, the primer additionally comprises a sense or an antisense sequence of a double-stranded promoter for a T7-type RNA polymerase selected from the group consisting of T7 RNA polymerase, T3 RNA polymerase, and SP6 RNA polymerase, and wherein the kit additionally comprises the RNA polymerase that can transcribe RNA using said promoter.

In some embodiments the kit additionally comprises: (i) a oligonucleotide sequence tag template; and (ii) a DNA polymerase selected from the group consisting of a DNA-dependent DNA polymerase and an RNA-dependent DNA polymerase that can extend the 3′-end of a nucleic acid that is annealed to the oligonucleotide sequence tag template. In some of these embodiments, the kit additionally comprises: (i) an oligonucleotide that is complementary to the oligonucleotide sequence tag template; and (ii) a T7-type RNA polymerase selected from the group consisting of T7 RNA polymerase, T3 RNA polymerase, and SP6 RNA polymerase; wherein the oligonucleotide sequence tag template and the oligonucleotide that is complementary to the oligonucleotide sequence tag template together encode the complete sequence of a promoter that can be used for transcription by said T7-type RNA polymerase.

In many cases, a kit of the present invention will contain one or more components selected from the group consisting of: (i) a negative control comprising an RNA having a 5′-triphosphate or a 5′-cap; (ii) a negative control comprising an RNA or a DNA having a 5′-hydroxyl group; (iii) a positive control comprising an RNA having a 5′-monophosphate; (iv) a solution of LiCl; (v) a ribonuclease H enzyme; and (vi) a polynucleotide kinase. The ribonuclease H(RNase H) can be used, together with a DNA oligonucleotide that anneals to a specific sequence of an mRNA molecule, to cleave the mRNA in a particular region prior to adding sequence tag by extension of the 3′-end of the RNase H-cleaved mRNA having a 5′-triphosphate or a 5′ cap using an oligonucleotide sequence tag template as a template. The polynucleotide kinase can be used to phosphorylate the 5′-end of degraded RNA molecules having a 5′-hydroxyl group in order to generate 5′-monophosphorylated RNA substrates that can digested using a 5′-to-3′ exoribonuclease of the invention.

Some kits of the invention will also comprise one or more components selected from the group consisting of: (i) a poly(A) polymerase; (ii) an oligo(dT)-containing primer; and (iii) an RNA-dependent DNA polymerase, such as, but not limited to, kits for enriching for prokaryotic mRNA. Some of such kits may also contain an RNA-dependent DNA polymerase and one or more primers selected from the group consisting of (i) a random primer, such as a random hexamer primer; (ii) an oligo(dT)-containing primer; and (iii) a primer that is complementary to a specific nucleic acid sequence. Some of such kits may also comprise: (i) a oligonucleotide sequence tag template for adding a tag sequence to the 3′-end of mRNA having a 5′-triphosphate or a 5′-cap; and (ii) a DNA polymerase selected from the group consisting of a DNA-dependent DNA polymerase and an RNA-dependent DNA polymerase that can extend the 3′-end of the mRNA using the oligonucleotide sequence tag template annealed to the mRNA as a template.

The kit of the invention can also comprise: (i) an oligonucleotide sequence tag template for adding a tag sequence to the 3′-end of mRNA having a 5′-triphosphate or a 5′-cap; and (ii) a DNA polymerase selected from the group consisting of a DNA-dependent DNA polymerase and an RNA-dependent DNA polymerase that can extend the 3′-end of the mRNA using the oligonucleotide sequence tag template annealed to the mRNA as a template. The DNA polymerase can be an RNA-dependent DNA polymerase, or, if a DNA-dependent DNA polymerase is used for adding the tag sequence to the 3′-end of the mRNA, the kit additionally contains an RNA-dependent DNA polymerase that can synthesize cDNA from a primer using the mRNA as a template.

In some embodiments of the invention, the oligonucleotide sequence tag template can additionally encode either a sense or an antisense strand of a double-stranded promoter for a T7-type RNA polymerase selected from the group consisting of T7 RNA polymerase, T3 RNA polymerase, and SP6 RNA polymerase, and in those embodiments, the kit can additionally comprise the RNA polymerase that can transcribe RNA using said promoter.

Still further, a kit of the invention can also comprise a poly(A) polymerase for tailing of prokaryotic mRNA, and an oligo(dT)-containing primer and an RNA-dependent DNA polymerase for synthesis of cDNA. The kit can also contain an oligonucleotide sequence tag template (for adding a sequence tag to the 3′-end of first-strand cDNA); an oligonucleotide that is complementary to the oligonucleotide sequence tag template; wherein the oligonucleotide sequence tag template and the oligonucleotide complementary to the oligonucleotide sequence tag template together can encode the complete sequence of a promoter for a T7-type RNA polymerase. Thus, in some such embodiments, the kit can also contain a T7-type RNA polymerase selected from the group consisting of a T7 RNA polymerase, a T3 RNA polymerase, and an SP6 RNA polymerase, wherein the T7-type RNA polymerase can use the promoter encoded by the combination of the oligonucleotide sequence tag template and the oligonucleotide complementary to the oligonucleotide sequence tag template.

Still other kits of the invention are useful for removing intact prokaryotic or eukaryotic ribosomal RNA (rRNA) of a size having a svedburg unit greater than about 10S from a biological sample. These kits can comprise: (i) a solution of purified and stabilized 5′-to-3′ exoribonuclease that is free of contaminating S. cerevisiae protein; (ii) a concentrated solution of a reaction buffer for providing a IX reaction buffer in which the exoribonuclease is active; and (iii) a positive control rRNA, selected from the group consisting of a 16S and a 23S prokaryotic rRNA, and an 18S, a 26S and a 28S eukaryotic rRNA. In a preferred embodiment of this aspect of the invention, the 5′-to-3′ exoribonuclease is a wild-type or recombinant Saccharomyces cerevisiae Xrn1p/5′ exoribonuclease 1.

In addition to the kits described above, it should be understood that the present invention provides a wide variety of kits for use in an equally wide variety of applications. In some preferred embodiments, the kits comprise a 5′-phosphate-dependent nucleic acid exonuclease (e.g., in a form, concentration, etc. suitable for use in the methods described herein). In other preferred embodiments, the kits comprise or consist of a 5′-phosphate-dependent nucleic acid exonuclease and one or more other components with which the 5′-phosphate-dependent nucleic acid exonuclease might be used directly or indirectly, either alone together or with yet other components. The one or more components may be any component involved in any of the methods described herein or in any of the methods described in the references or patents cited herein. The one or more components include, but are not limited to, buffers, salts, enzymes, proteins, antibodies, nucleic acid molecules (e.g., primers, probes, antisense oligonucleotides, RNA, DNA, etc.), nucleotides, control reagents, instructions, solid surfaces (e.g., beads, microarray components, etc.), detection reagents (e.g., fluorescent, luminescent, colorimetric, etc.), general laboratory equipment (tubes, multiwell plates, etc.), software, vectors or vector components, cell culture reagents and materials, and the like.

### DESCRIPTION OF THE FIGURE

FIG. 1 shows the amino acid (SEQ ID NO:1) and nucleic acid (SEQ ID NO:2) sequences of Saccharomyces cerevisiae Xrn1p.

### DEFINITIONS

The present invention will be understood and interpreted based on the definitions of terms as defined below.

A “5′-to-3′ exoribonuclease” or a “5′ exoribonuclease” or a “5′ Xrn1p exoribonuclease” or a “5′ exonuclease” or a “5′-phosphate-dependent exonuclease” or a “5′-phosphate-dependent nucleic acid exonuclease” or a “5 nuclease” of the present invention comprises an 5′-exonuclease that has greater than 20-fold more 5′-to-3′ exonuclease activity for a single-stranded RNA substrate that has a 5′-monophosphorylated terminus than for the same RNA substrate that has a 5′-triphosphorylated or 5′-capped terminus, or, any 5′-to-3′ exonuclease that has a relative activity in digesting a particular defined-sequence single-stranded RNA substrate with a 5′-triphosphate or a 5′-cap that is less than 5% of the activity for an RNA substrate having the same sequence but with a 5′-monophosphate. Enzyme activity of a 5′ exonuclease of the invention can be measured using a number of different methods. Without limitation, suitable methods that can be used for assaying activity and determining relative activity using RNA substrates with a 5′-triphosphate, a 5′-cap, or a 5′-monophosphate are described by Stevens and Poole (J. Biol. Chem., 270: 16063, 1995).

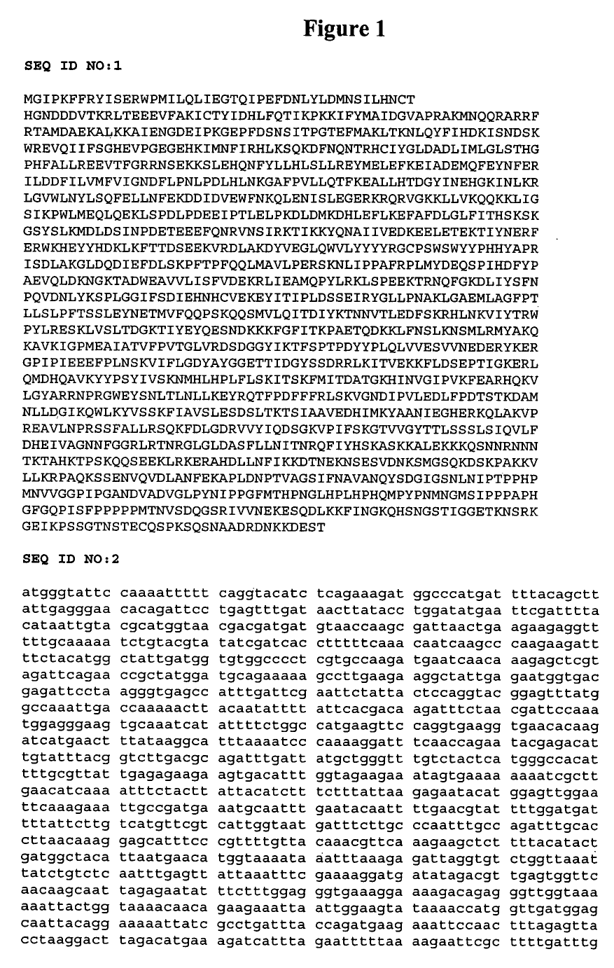

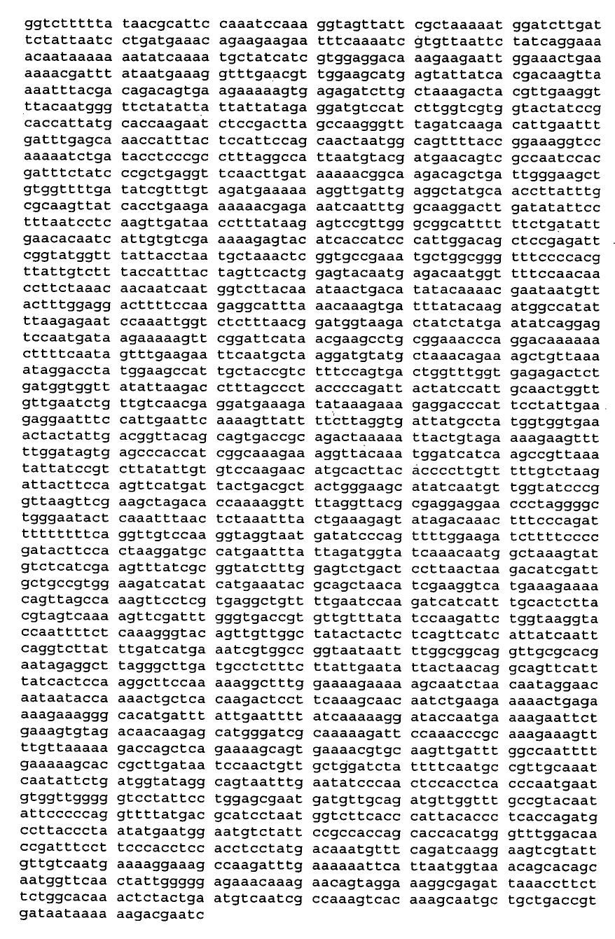

Preferred embodiments of compositions, kits and methods of the invention employ Xrn1p/5′ exoribonuclease 1, including Saccharomyces cerevisiae Xrn1p/5′ exoribonuclease 1, and/or functional variants and homologues thereof. Xrn1p was first identified and characterized in Saccharomyces cerevisiae, but the activity has been reported in numerous organisms, including humans. In preferred embodiments of the present invention, the 5′-to-3′ exoribonuclease comprises a solution of the enzyme that has been purified so as to be free of contaminating enzymes with activity on nucleic acids and Saccharomyces cerevisiae proteins. In some preferred embodiments, the 5′-to-3′ exoribonuclease is obtained by expression of the Saccharomyces cerevisiae XRN1 gene that has been cloned in a plasmid, and then replicated and expressed in Esherichia coli cells, since the Xrn1p/5′ exoribonuclease I obtained from such a recombinant source is of a higher purity, free from contaminating enzymatic activities, and generally at a higher enzyme concentration than is obtained from non-recombinant sources. In preferred embodiments of the invention, the 5′-to-3′ exoribonuclease is “stabilized”, by which we mean that the 5′-to-3′ exoribonuclease is sufficiently pure of proteases and other contaminants which contribute to degradation and loss of enzyme activity and that the 5′-to-3′ exoribonuclease is provided in a formulation of enzyme storage buffer in which there is no significant loss of activity during storage at −20 degrees C. for six months. One suitable enzyme storage buffer for providing a stabilized 5′-to-3′ exoribonuclease comprises a 50% glycerol solution containing 50 mM Tris-HCL (pH 7.5), 100 mM NaCl, 100 mM EDTA, 1 mM DTT and 0.1% of the non-ionic detergent Triton X-100. The Saccharomyces cerevisiae purified yeast enzyme was initially found to be a 160-kDa protein but, based on its gene sequence, the actual size is closer to 175 kDa. The amino acid (SEQ ID NO:1) and nucleic acid (SEQ ID NO:2) sequences of Saccharomyces cerevisiae Xrn1p are shown in FIG. 1. “Xrn1p” generally refers to the protein, whereas “XRN1” generally refers to the gene; however, the terms “Xrn1p” and “Xrn1p/5′ exoribonuclease I” are used interchangeably herein and the term “Xrn1p”, as used herein, refers to both the protein and gene unless indicated otherwise. The enzyme is a processive exoribonuclease, requiring a divalent cation, and generally is stimulated by monovalent cations. The enzyme acts on a variety of substrates, including homopolymers, rRNA, and oligoadenylates. The enzyme also has some DNase activity. Single-stranded molecules are the preferred substrate. The XRN1 gene of Saccharomyces cerevisiae is identical to a gene (DST2, SEP1) encoding a DNA transferase and to genes involved in nuclease fusion, KEM1, and plasmid stability, RAR5 (see e.g., Larimer et al., Gene 120:51, 1992). Another name for this gene is Stpβ (see e.g., Heyer et al., Mol. Cell Biol. 15:2728, 1995).

Without being bound by theory, sequence comparison of several Mg2+-dependent 5′-3′ exonucleases from bacteriophages, prokaryotes, and eukaryotes (Solinger et al., Mol. Cell. Biol. 19:5930, 1995) revealed some highly conserved amino acid residues. In the crystal structure of phage T4 RNase H, these residues are clustered in the proposed active site. In particular, some conserved aspartic (D) and glutamic (E) acid residues coordinate two Mg2+ ions in the reactive center of the exonuclease and are believed to play a crucial role in catalysis. Mutation of D206 and D208 of S. cerevisiae Xrn1p abolished exonuclease activity in the mutants.

Moreover, variant forms of Xrn1p are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide variants of Xrn1p disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (e.g., Stryer ed., Biochemistry, pg. 17-21, 2nd ed, WH Freeman and Co., 1981). It can be readily determined whether a change in the amino acid sequence of a peptide results in a functional polypeptide by assessing the ability of the variant peptide to function in a fashion similar to the wild-type protein. Peptides having more than one replacement can readily be tested in the same manner.

More rarely, a variant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

Variants may be produced by methods such as directed evolution or other techniques for producing combinatorial libraries of variants, described in more detail below. In still other embodiments of the present invention, the nucleotide sequences of the present invention may be engineered in order to alter an Xrn1p coding sequence including, but not limited to, alterations that modify the cloning, processing, localization, secretion, and/or expression of the gene product. For example, mutations may be introduced using techniques that are well known in the art (e.g., site-directed mutagenesis to insert new restriction sites, alter glycosylation patterns, or change codon preference, etc.).

Still other embodiments of the present invention provide mutant or variant forms of Xrn1p. It is possible to modify the structure of a peptide having an activity of Xrn1p for such purposes as enhancing activity, or stability (e.g., ex vivo shelf life, and/or resistance to proteolytic degradation in vivo). Such modified peptides are considered functional equivalents of peptides having an activity of the subject Xrn1p proteins as defined herein. A modified peptide can be produced in which the amino acid sequence has been altered, such as by amino acid substitution, deletion, or addition.

Moreover, as described above, variant forms (e.g., mutants) of the subject Xrn1p proteins are also contemplated as being equivalent to those peptides and DNA molecules that are set forth in more detail. For example, as described above, the present invention encompasses mutant and variant proteins that contain conservative or non-conservative amino acid substitutions.

This invention further contemplates a method of generating sets of combinatorial mutants of the present Xrn1p proteins, as well as truncation mutants, and is especially useful for identifying potential variant sequences (i.e., mutants) that are functional in exoribonuclease activity. The purpose of screening such combinatorial libraries is to generate, for example, novel Xrn1p variants that have improved or altered exoribonuclease activity.

Therefore, in some embodiments of the present invention, Xrn1p variants are engineered by the present method to provide altered (e.g., increased or decreased) exoribonuclease activity. In other embodiments, Xrn1p variants are engineered to provide heat-stable (i.e., “thermostable”) or heat-labile exoribonuclease activity for particular applications. In other embodiments of the present invention, combinatorially-derived variants are generated which have substrate variability different than that of a naturally occurring Xrn1p. Such proteins, when expressed from recombinant DNA constructs, find use in the methods described herein.

Still other embodiments of the present invention provide Xrn1p variants that have intracellular half-lives dramatically different than the corresponding wild-type protein. For example, the altered protein can be rendered either more stable or less stable to proteolytic degradation or other cellular process that result in destruction of, or otherwise inactivate Xrn1p. Such variants, and the genes which encode them, can be utilized to alter the location of Xrn1p expression by modulating the half-life of the protein. For instance, a short half-life can give rise to more transient Xrn1p biological effects and, when part of an inducible expression system, can allow tighter control of Xrn1p levels within the cell.

In still other embodiments of the present invention, Xrn1p variants are generated by the combinatorial approach to act as antagonists, in that they are able to interfere with the ability of the corresponding wild-type protein to regulate cell function.

In some embodiments of the combinatorial mutagenesis approach of the present invention, the amino acid sequences for a population of Xrn1p homologs, variants or other related proteins are aligned, preferably to promote the highest homology possible. Such a population of variants can include, for example, Xrn1p homologs from one or more species, or Xrn1p variants from the same species but which differ due to mutation or polymorphisms. Amino acids that appear at each position of the aligned sequences are selected to create a degenerate set of combinatorial sequences.

In a preferred embodiment of the present invention, the combinatorial Xrn1p library is produced by way of a degenerate library of genes encoding a library of polypeptides which each include at least a portion of potential Xrn1p protein sequences. For example, a mixture of synthetic oligonucleotides can be enzymatically ligated into gene sequences such that the degenerate set of potential Xrn1p sequences are expressible as individual polypeptides, or alternatively, as a set of larger fusion proteins (e.g., for phage display) containing the set of Xrn1p sequences therein.

There are many ways by which the library of potential Xrn1p homologs and variants can be generated from a degenerate oligonucleotide sequence. In some embodiments, chemical synthesis of a degenerate gene sequence is carried out in an automatic DNA synthesizer, and the synthetic genes are ligated into an appropriate gene for expression. The purpose of a degenerate set of genes is to provide, in one mixture, all of the sequences encoding the desired set of potential Xrn1p sequences. The synthesis of degenerate oligonucleotides is well known in the art (See e.g., Narang, Tetrahedron Lett., 39:39, 1983; Itakura et al., Recombinant DNA, in Walton (ed.), Proceedings of the 3rd Cleveland Symposium on Macromolecules, Elsevier, Amsterdam, pp 273-289, 1981; Itakura et al., Annu. Rev. Biochem., 53:323, 1984; Itakura et al., Science 198:1056, 1984; Ike et al., Nucl. Acid Res., 11:477, 1983). Such techniques have been employed in the directed evolution of other proteins (See e.g., Scott et al., Science 249:386 [1980]; Roberts et al., Proc. Natl. Acad. Sci. USA 89:2429 [1992]; Devlin et al., Science 249: 404 [1990]; Cwirla et al., Proc. Natl. Acad. Sci. USA 87: 6378 [1990]; as well as U.S. Pat. Nos. 5,223,409, 5,198,346, and 5,096,815; each of which is incorporated herein by reference).

It is contemplated that the Xrn1p nucleic acids (e.g., SEQ ID NO:2, and fragments and variants thereof) can be utilized as starting nucleic acids for directed evolution. These techniques can be utilized to develop Xrn1p variants having desirable properties such as increased, decreased, or altered exoribonuclease activity.

In some embodiments, artificial evolution is performed by random mutagenesis (e.g., by utilizing error-prone PCR to introduce random mutations into a given coding sequence). This method requires that the frequency of mutation be finely tuned. As a general rule, beneficial mutations are rare, while deleterious mutations are common. This is because the combination of a deleterious mutation and a beneficial mutation often results in an inactive enzyme. The ideal number of base substitutions for targeted gene is usually between 1.5 and 5 (Moore and Arnold, Nat. Biotech., 14, 458, 1996; Eckert and Kunkel, PCR Methods Appl., 1: 17-24, 1991; Caldwell and Joyce, PCR Methods Appl., 2:28, 1992; and Zhao and Arnold, Nuc. Acids Res. 25:1307, 1997). After mutagenesis, the resulting clones are selected for desirable activity (e.g., screened for Xrn1p activity). Successive rounds of mutagenesis and selection are often necessary to develop enzymes with desirable properties. It should be noted that only the useful mutations are carried over to the next round of mutagenesis.

In other embodiments of the present invention, the polynucleotides of the present invention are used in gene shuffling or sexual PCR procedures (e.g., Smith, Nature, 370:324, 1994; U.S. Pat. Nos. 5,837,458; 5,830,721; 5,811,238; 5,733,731; all of which are herein incorporated by reference). Gene shuffling involves random fragmentation of several mutant DNAs followed by their reassembly by PCR into full length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations present in different clones becoming mixed and accumulating in some of the resulting sequences. Multiple cycles of selection and shuffling have led to the functional enhancement of several enzymes (Stemmer, Nature, 370:398, 1994; Stemmer, Proc. Natl. Acad. Sci. USA, 91:10747, 1994; Crameri et al., Nat. Biotech., 14:315, 1996; Zhang et al., Proc. Natl. Acad. Sci. USA, 94:4504, 1997; and Crameri et al., Nat. Biotech., 15:436, 1997).

A wide range of techniques are known in the art for screening gene products of combinatorial libraries made by point mutations, and for screening cDNA libraries for gene products having a certain property. Such techniques will be generally adaptable for rapid screening of the gene libraries generated by the combinatorial mutagenesis or recombination of Xrn1p homologs or variants. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

Homologues and similar proteins that find use in the compositions and methods of the present invention include proteins and extracts containing proteins from a variety of organisms. For example, Rat1p/5′ exonuclease 2 and homologues DHp1p and Dhm1p in mouse and humans may be used in the compositions and methods of the present invention. Work with crude extracts shows a homologue in Xenopus (see e.g., Furuichi et al., Nature 266:235, 1977) and wheat germ (see e.g., Shimotohno et al., Proc. Natl. Acad. Sci. USA 74:2734, 1977). Stevens et al. showed purification of a 5′ exoribonuclease from human placental nuclei (Nucl. Acids Res., 15:695, 1987). Heyer et al., show that antibodies against the yeast Xrn1 protein also interact with the homologue protein in Drosophila, Xenopus, and mouse (Mol. Cell. Biol., 15:2728, 1995).

Fragments of the nucleic acids and proteins of the present invention may also be used, so long as the fragments encode or possess the desired enzymatic activity. Page et al. (Nucl. Acids Res., 26:3707, 1998), herein incorporated by reference in its entirety, provide deletion analysis demonstrating that the C-terminus is dispensable for exonuclease function.

A “unit” of exoribonuclease is the amount of enzyme required to convert 1 μg of E. coli ribosomal RNA (i.e., 16S and/or 23S rRNA) to acid-soluble form in sixty minutes at 30° C. under standard reaction assay conditions (e.g., 50 mM Tris-HCl, pH 8.0, 2 mM MgCl₂, and 0.1 M NaCl).

The symbol “S” with respect to a ribosomal RNA, such as “16S” or “23S” rRNA, or another macromolecule, designates a “svedberg unit” (named for Theodor Svedberg), which is a unit used to measure the sedimentation rate of a molecule in a colloidal suspension and which indicates the molecular weight of the molecule, with a svedberg unit being equal to 10⁻¹³ second.

The term “gene” as used herein, refers to a DNA sequence that comprises control and coding sequences necessary for the production of a polypeptide or protein precursor or other encoded molecule (e.g., rRNA, tRNA). The polypeptide can be encoded by a full-length coding sequence or by any portion of the coding sequence, as long as the desired protein activity is retained.

“Nucleoside”, as used herein, refers to a compound consisting of a purine (guanine (G) or adenine (A)) or pyrimidine (thymine (T), uridine (U), or cytidine (C)) base covalently linked to a pentose sugar, whereas “nucleotide” refers to a nucleoside phosphorylated at one of the hydroxyl groups of the pentose sugar.

A “cap” is a modified guanine nucleotide that is joined to the 5′-end of the 5′ nucleotide of a primary eukaryotic mRNA molecule. A common cap (sometimes referred to as “standard cap”) has a structure designated as m⁷G[5′]ppp[5′]N, in which “p” represents a phosphate group, “G” represents a guanosine nucleoside, “m⁷” represents a methyl group on the 7-position of guanine (in this example, on the guanine base of the cap nucleotide), and “[5′]” indicates the position at which the “p” is joined to the ribose of each respective nucleoside (in this example, indicating that the 5′-carbon of the ribose of the cap is joined via a triphosphate group to the 5′-carbon of the ribose of the first nucleoside (“N”) of the mRNA. In addition to this “standard” cap, a variety of other naturally-occurring and synthetic cap analogs are known in the art.

A “nucleic acid” or a “polynucleotide”, as used herein, is a covalently linked sequence of nucleotides in which the 3′ position of the sugar moiety of one nucleotide is joined by a phosphodiester group to the 5′ position of the sugar moiety of the next nucleotide, and in which the nucleotide residues (bases) are linked in specific sequence; i.e., a linear order of nucleotides. An “oligonucleotide”, as used herein, is a short polynucleotide or a portion of a polynucleotide. An oligonucleotide typically contains a sequence of about two to about one hundred bases, although longer molecules may also be encompassed by this term. The word “oligo” is sometimes used in place of the word “oligonucleotide”.

An “oligonucleotide sequence tag template” or a “splice template oligo” or a “template switch oligo” is an oligonucleotide that complexes with a single-stranded target nucleic acid and is used as a template to extend the 3′-terminus of one or more target sequences in order to add a specific sequence or a “sequence tag”. The 3′-portion of an oligonucleotide sequence tag template or a splice template is sufficiently complementary to the 3′-terminus of the target sequence that is to be extended to anneal thereto. A DNA- or RNA-dependent DNA polymerase is then used to extend the target nucleic acid molecule using the sequence in the 5′-portion of the oligonucleotide sequence tag template or splice template oligo as a template. The extension product of the primer-extended molecule has the specific sequence at its 3′-terminus that is complementary to the sequence in the 5′-portion of the oligonucleotide sequence tag template or splice template oligo. With respect to the present invention, an oligonucleotide sequence tag template can be used to add a sequence tag to the 3′-end of a nucleic acid, including an RNA or DNA molecule, as described in U.S. Patent Application No. 2005/015333 of Roy R. Sooknanan, which is incorporated herein by reference.

One embodiment of an oligonucleotide sequence tag template or splice template oligo of the present invention is a “promoter sequence tag template” or “promoter splice template oligo.” A promoter sequence tag template or promoter splice template oligo comprises a sequence in its 3′-portion that is sufficiently complementary to the 3′-end of the target sequence to anneal thereto and a sequence in it 5′-portion that is complementary to a sequence comprising a single-stranded transcription promoter of the invention. Thus, a promoter sequence tag template or promoter splice template oligo can provide a template for synthesis of a sequence comprising a transcription promoter at the 3′-end of first-strand cDNA obtained either by RNA-dependent DNA polymerase (or “reverse transcriptase”) primer extension of a target sequence comprising an RNA target nucleic acid, such as, but not limited to, an mRNA target, or by DNA polymerase primer extension of a target sequence comprising DNA or RNA.

A “ligation splint oligo” or “ligation splint” is an oligo that is used to provide an annealing site or a “ligation template” for joining two ends of one nucleic acid (i.e., “intramolecular joining”) or two ends of two nucleic acids (i.e., “intermolecular joining”) using a ligase or another enzyme with ligase activity. The ligation splint holds the ends adjacent to each other and “creates a ligation junction” between the 5′-phosphorylated and a 3′-hydroxylated ends that are to be ligated.

Nucleic acid molecules are said to have a “5′-terminus” (5′ end) and a “3′-terminus” (3′ end) because nucleic acid phosphodiester linkages occur at the 5′ carbon and 3′ carbon of the sugar moieties of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A terminal nucleotide, as used herein, is the nucleotide at the end position of the 3′- or 5′-terminus.

Nucleic acid molecules are said to have “5′ ends” and “3′ ends” because mononucleotides are joined in one direction via a phosphodiester linkage to make oligonucleotides, in a manner such that a phosphate on the 5′-carbon of one mononucleotide sugar moiety is joined to an oxygen on the 3′-carbon of the sugar moiety of its neighboring mononucleotide. Therefore, an end of an oligonucleotide referred to as the “5′ end” if its 5′ phosphate is not linked to the oxygen of the 3′-carbon of a mononucleotide sugar moiety and as the “3′ end” if its 3′ oxygen is not linked to a 5′ phosphate of the sugar moiety of a subsequent mononucleotide.

Polypeptide molecules are said to have an “amino terminus” (N-terminus) and a “carboxy terminus” (C-terminus) because peptide linkages occur between the backbone amino group of a first amino acid residue and the backbone carboxyl group of a second amino acid residue.

As used herein, the terms “complementary” or “complementarity” are used in reference to a sequence of nucleotides related by the base-pairing rules. For example, the sequence 5′-A-G-T-3′, is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon hybridization of nucleic acids.

As used herein, the term “probe” refers to an oligonucleotide (i.e., a sequence of nucleotides), whether occurring naturally (e.g., as in a purified restriction digest) or produced synthetically, recombinantly or by PCR amplification, which is capable of hybridizing to another oligonucleotide of interest. A probe may be single-stranded or double-stranded (e.g., and rendered single-stranded or partially single-stranded in use). Probes are useful in the detection, identification and isolation of particular gene sequences. It is contemplated that a probe used in the present invention can be labeled with any “reporter molecule,” so that it is detectable in a detection system, including, but not limited to enzyme (i.e., ELISA, as well as enzyme-based histochemical assays), visible, fluorescent, radioactive, and luminescent systems. It is not intended that the present invention be limited to any particular detection system or label. The terms “reporter molecule” and “label” are used herein interchangeably. In addition to probes, primers and deoxyribonucleoside triphosphates may contain labels; these labels may comprise, but are not limited to, ³²P, ³³P, ³⁵S, enzymes, or visible, luminescent, or fluorescent molecules (e.g., fluorescent dyes).

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical or similar properties as compared to the native form of the protein. Variants of the native sequence may also be made to, for example, improve expression, purification, or other desired properties of the polypeptide.

As used herein in reference to an amino acid sequence or a protein, the term “portion” (as in “a portion of an amino acid sequence”) refers to fragments of that protein. The fragments may range in size from four amino acid residues to the entire amino acid sequence minus one amino acid. Particularly preferred fragments retain one or more of the enzymatic activities associated with a whole protein (e.g., 5′ exoribonuclease activity).

As used herein, the term “fusion protein” refers to a chimeric protein containing the protein of interest (e.g., 5′ exoribonuclease 1 and fragments thereof) joined to an exogenous protein fragment (e.g., the fusion partner which contains a non-xrn-1 protein). The fusion partner may enhance the solubility of xrn-1 protein as expressed in a host cell, may provide an affinity tag to allow purification of the recombinant fusion protein from the host cell or culture supernatant, or both. If desired, the fusion protein may be removed from the protein of interest (e.g., xrn-1 or fragments thereof) by a variety of enzymatic or chemical means know to the art.

The term “5′ to 3′ exonuclease activity” refers to the presence of an activity in a protein that is capable of removing nucleotides from the 5′ end of an oligonucleotide. 5′ to 3′ exonuclease activity may be measured using any of the assays provided herein or known in the art. Some enzymes contain 5′ to 3′ “exoribonuclease” activity, the ability to remove nucleotides from the 5′ end of ribonucleic acid molecules. Some enzymes that are traditionally called “exoribonucleases” may also have 5′-exonuclease activity against DNA substrates. For example, enzymes of the present invention, while historically named “exoribonucleases”, have 5′-exonuclease activity on DNA substrates that have a 5′-phosphate, meaning a phosphate group on the 5′-position of the deoxyribose sugar of the nucleotide at the 5′-end of the DNA. Thus, it is understood that such exoribonucleases (e.g., Xrn15′ exoribonuclease 1), unless specified otherwise, have exonuclease activity for both RNA and DNA substrates.

The terms “cell,” “cell line,” “host cell,” as used herein, are used interchangeably, and all such designations include progeny or potential progeny of these designations. The words “transformants” or “transformed cells” include the primary transformed cells derived from that cell without regard to the number of transfers. All progeny may not be precisely identical in DNA content, due to deliberate or inadvertent mutations. Nonetheless, mutant progeny that have the same functionality as screened for in the originally transformed cell are included in the definition of transformants.

Nucleic acids are known to contain different types of mutations. A “point” mutation refers to an alteration of a base nucleotide at a single nucleotide position in the sequence compared to the wild type sequence. Mutations may also refer to insertion or deletion of one or more bases, so that the nucleic acid sequence differs from the wild-type sequence.

The term “homology” refers to a degree of complementarity of one nucleic acid sequence with another nucleic acid sequence. There may be partial homology or complete homology (i.e., complementarity). A partially complementary sequence is one that at least partially inhibits a completely complementary sequence from hybridizing to a target nucleic acid and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target under conditions of low stringency. This is not to say that conditions of low stringency are such that nonspecific binding is permitted; low stringency conditions require that the binding of two sequences to one another be a specific (i.e., selective) interaction. The absence of nonspecific binding may be tested by the use of a second target that lacks complementarity or that has only a low degree of complementarity (e.g., less than about 30% complementarity). In the case in which specific binding is low or non-existent, the probe will not hybridize to a nucleic acid target.

When used in reference to a double-stranded nucleic acid sequence such as a cDNA or a genomic clone, the term “substantially homologous” refers to any probe which can hybridize to either or both strands of the double-stranded nucleic acid sequence under conditions of low stringency as described herein.

As used herein, the terms “hybridization” or “annealing” are used in reference to the pairing of complementary nucleic acid strands. Hybridization and the strength of hybridization (i.e., the strength of the association between nucleic acid strands) is impacted by many factors well known in the art including the degree of complementarity between the nucleic acids, stringency of the conditions involved affected by such conditions as the concentration of salts, the Tₘ (melting temperature) of the formed hybrid, the presence of other components (e.g., the presence or absence of polyethylene glycol or betaine), the molarity of the hybridizing strands and the G:C content of the nucleic acid strands.

The term “isolated” when used in relation to a nucleic acid, as in “isolated polynucleotide” or “isolated oligonucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; and a specific RNA sequences (e.g., a specific mRNA sequence encoding a specific protein), is found in the cell as a mixture with numerous other mRNAs that encode a multitude of proteins. The isolated polynucleotide or nucleic acid or oligonucleotide may be present in single-stranded or double-stranded form. When an isolated polynucleotide or nucleic acid is to be utilized to express a protein, the polynucleotide contains at a minimum, the sense or coding strand (i.e., the polynucleotide may be single-stranded), but may contain both the sense and anti-sense strands (i.e., the polynucleotide may be double-stranded).

As used herein, the term “purified” or “to purify” means the result of any process that removes some of a contaminant from the component of interest, such as a protein or nucleic acid. The percent of a purified component is thereby increased in the sample.

As used herein, the term “enzyme” refers to molecules or molecule aggregates that are responsible for catalyzing chemical and biological reactions. Such molecules are typically proteins, but can also comprise short peptides, RNAs, ribozymes, antibodies, and other molecules. A molecule that catalyzes chemical and biological reactions is referred to as “having enzyme activity” or “having catalytic activity.”

In addition to 5′-phosphate-dependent exonucleases, a variety of other enzymes are used in the methods of the present invention. In general, the invention comprises use of any enzyme from any source that has an activity that is equivalent to an enzyme referred to in a method so long as the enzyme functions in the same way in the particular method. By way of example, but not of limitation, an RNA-dependent DNA polymerase can comprise an AMV reverse transcriptase; an MMLV reverse transcriptase; SuperScript I, SuperScript II, SuperScript III, or AMV ThermoScript reverse transcriptase (INVITROGEN); or MonsterScript reverse transcriptase (EPICENTRE), or it can comprise another enzyme that has similar activity in the method; a polynucleotide kinase can comprise T4 polynucleotide kinase or it can comprise another enzyme that has similar activity in the method; a poly(A) polymerase can comprise an E. coli poly(A) polymerase encoded by the pcnB gene or it can comprise another enzyme that has similar activity in the method; a ribonuclease H can comprise E. coli RNase H or a Thermus RNase H (e.g., Hybridase RNase H from EPICENTRE) or it can comprise another enzyme that has similar activity in the method; a pyrophosphatase can be tobacco acid pyrophosphatase or it can comprise another enzyme that has similar activity in the method; and a DNA phosphatase can comprise APex Alkaline Phosphatase (EPICENTRE) or shrimp alkaline phosphatase or it can comprise another enzyme that has similar activity in the method. Methods and reaction conditions for use of these and other enzymes that can be used are well known in the art.

An “RNA amplification method” according to the present invention is a method that synthesizes an RNA product and that results in an increase in the number of copies of an RNA sequence or of its complementary sequence compared to the number of copies of the sequence present in a sample. A number of RNA amplification methods are known in the art that comprise steps for joining an RNA polymerase promoter to either the 5′-end of the cDNA or the 3′-end of cDNA that is made by reverse transcription of mRNA using an RNA-dependent DNA polymerase to extend a primer, such as, but not limited to an oligo(dT)-containing primer, which methods result in synthesis of amplified antisense RNA or amplified sense RNA, respectively, by subsequent in vitro transcription (e.g., using T7, T3, or SP6 RNA polymerase). Kits and methods of the present invention that comprise RNA amplification can comprise any compositions or method, including without limitation, any compositions or methods known in the art. By way of example, but not of limitation, a method that uses an oligo(dT) promoter primer to synthesize an antisense RNA (aRNA), such as methods described by Van Gelder, R. N., et al. (Proc. Natl. Acad. Sci. USA 87: 1663, 1990) can be used; such methods are sometimes referred to as “Eberwine-type” amplification methods herein. Kits for this purpose are commercially available and can be used, including 1-round and 2-round amplification kits such as various 1-round and 2-round TargetAmp™ Aminoallyl-aRNA Amplification Kits or TargetAmp™ aRNA Amplification Kits available from EPICENTRE, or MessageAmp kits available from Ambion (Austin, Tex.). Still further, a sense RNA amplification method that attaches a promoter to the 3′-end of first-strand cDNA for synthesis of amplified sense RNA, such as methods described by Che, S. and Ginsberg, S. D (Laboratory Investigation 84: 131, 2004) and by Ginsberg and Che (Neurochemical Res. 27: 981, 2002; and PCT Internatl. Publication No. WO 02/065093), can be used in a method or kit of the present invention. The BD SMART mRNA amplification kit (BD Biosciences Clontech, Palo Alto, Calif.) is an example of a commercial kit of a type that can be used for sense RNA amplification in methods and kits of the present invention. Still another RNA amplification method that can be used in the methods of the present invention uses a oligonucleotide sequence tag template to add a tag sequence to the 3′-end of a target sequence comprising mRNA or first-strand cDNA, which tag sequence can be further used to add a promoter for synthesis of RNA corresponding to the target sequence, as described in U.S. Patent Application No. 2005/015333 of Roy R. Sooknanan, which is incorporated herein by reference. Examples of other methods for RNA amplification that can be used are described in: Murakawa et al., DNA 7:287-295, 1988; Phillips and Eberwine, Methods in Enzymol. Suppl. 10:283-288, 1996; Ginsberg et al., Ann. Neurol. 45:174-181, 1999; Ginsberg et al., Ann. Neurol. 48:77-87, 2000; VanGelder et al., Proc. Natl. Acad. Sci. USA 87:1663-1667, 1990; Eberwine et al., Proc. Natl. Acad. Sci. USA 89:3010-3014, 1992; U.S. Pat. Nos. 5,021,335; 5,168,038; 5,545,522; 5,514,545; 5,716,785; 5,891,636; 5,958,688; 6,291,170; and PCT Patent Applications WO 00/75356 and WO 02/065093, which is incorporated herein by reference.

The present invention is also not limited to RNA amplification methods that require synthesis of double-stranded cDNA. By way of example, but without limitation, the present invention also comprises RNA amplification methods and compositions as described in U.S. Patent Appln. No. 2004/0171041 that use an RNA polymerase that can synthesize RNA using single-stranded templates that are functionally joined to a single-stranded promoter, such as, but not limited to methods that use MiniV RNA polymerase (available from EPICENTRE in the MiniV™ In Vitro Transcription Kit); in these embodiments, a single-stranded promoter is joined to either the 5′-end of the cDNA or the 3′-end of cDNA that is made by reverse transcription of mRNA using an RNA-dependent DNA polymerase to extend a primer, resulting in synthesis of amplified antisense RNA or amplified sense RNA, respectively, by subsequent in vitro transcription of single-stranded DNA templates (e.g., using MiniV RNA polymerase).

A “T7-type RNA polymerase” as defined herein is a wild-type or mutant form of an RNA polymerase derived from a T7-type bacteriophage, including both phage-encoded enzymes and enzymes obtained by cloning the RNA polymerase gene in a DNA vector and expressing it in a bacterial or other cell. This is based on the fact that the genetic organization of all T7-type bacteriophage that have been examined has been found to be essentially the same as that of T7. Examples of T7-type bacteriophages according to the invention include, but are not limited to Escherichia coli phages T3, phi I, phi II, W31, H, Y, A1, 122, cro, C21, C22, and C23; Pseudomonas putida phage gh-1; Salmonella typhimurium phage SP6; Serratia marcescens phages IV; Citrobacter phage ViIII; and Klebsiella phage No. 11 (Hausmann, Current Topics in Microbiology and Immunology 75:77-109, 1976; Korsten et al., J. Gen. Virol. 43:57-73, 1975; Dunn, et al., Nature New Biology 230:94-96, 1971; Towle, et al., J. Biol. Chem. 250:1723-1733, 1975; Butler and Chamberlin, J. Biol. Chem. 257:5772-5778, 1982). Mutant RNAPs (Sousa et al., U.S. Pat. No. 5,849,546; Padilla, R and Sousa, R, Nucleic Acids Res., 15: e138, 2002; Sousa, R and Mukherjee, S, Prog Nucleic Acid Res Mol. Biol., 73: 1-41, 2003), such as, but not limited to, T7 RNAP Y639F mutant enzyme, T3 RNAP Y573F mutant enzyme, SP6 RNAP Y631F mutant enzyme, T7 RNAP having altered amino acids at both positions 639 and 784, T3 RNAP having altered amino acids at both positions 573 and 785, or SP6 RNAP having altered amino acids at both positions 631 and 779 can also be used in some embodiments of methods or assays of the invention. In particular, such mutant enzymes can corporate dNTPs and 2′-F-dNTPs, in addition to ddNTPs and certain other substrates, which are advantageous for synthesis of RNA molecules with specific properties and uses. Phage N4 mini-vRNAP, which has certain domains in common with T7-type RNAPs, and which is also an RNA polymerase of the present invention, is a transcriptionally active 1,106-amino acid domain of the N4 vRNAP, which corresponds to amino acids 998-2103 of N4 vRNAP (Kazmierczak, K. M., et al., EMBO J., 21: 5815-5823, 2002; U.S. Patent Application No. 20030096349, incorporated herein by reference) can be used. Alternatively, an N4 mini-vRNAP Y678F mutant enzyme (U.S. Patent Application No. 20030096349) can incorporate non-canonical nucleotides such as 2′-F-dNTPs. In order to carry out transcription, a RNA polymerase recognizes and binds to a DNA sequence of approximately 25 nucleotides in length called an “RNA polymerase promoter,” a “transcription promoter” or simply a “promoter,” and initiates transcription therefrom. In most cases, the promoter sequence is double-stranded. As used herein, the strand of a double-stranded promoter that is covalently joined to the template strand for synthesis of RNA is defined as the “sense strand” or “sense promoter sequence” and its complement is defined as the “anti-sense strand” or the “anti-sense promoter sequence.”

Still further, the term “RNA amplification method” according to the present invention also includes other methods, such as but not limited to methods described in U.S. Pat. Appln. No. 2004/0180361 of Dahl et al.; PCT Patent Publication Nos. WO 02/16639; WO 00/56877; and AU 00/29742 of Takara Shuzo Company; and in U.S. Pat. No. 6,251,639 and U.S. Pat. Appln. Nos. 2001/0034048; 2003/0017591; 2003/0087251; and 2003/0186234 of Kurn (all incorporated herein by reference), even if said methods comprise synthesis DNA products rather than RNA products corresponding to mRNA in the sample. Thus, the present invention also comprises embodiments in which a nucleic acid of interest (or a desired nucleic acid) obtained following treatment with a 5′-phosphate-dependent exonuclease of the present invention is amplified using any of said RNA amplification methods, including those that comprise synthesis of DNA amplification products.

A nucleic acid of interest (or a desired nucleic acid) obtained following treatment with a 5′-phosphate-dependent exonuclease of the present invention can also be amplified by PCR or reverse transcription-PCR (i.e., RT-PCR), including without limitation real-time PCR or real-time RT-PCR. Amplification of an mRNA of interest or a cDNA of interest by RT-PCR or PCR, respectively, is useful for validation of results of gene expression analysis using other methods, such as but not limited to, gene expression analysis results obtained by hybridization of labeled RNA or DNA corresponding to mRNA from a sample to nucleic acid arrays or microarrays.

“Rolling Circle Replication” and “Rolling Circle Transcription” are two other methods that can be used to amplify a nucleic acid of interest in a method of the present invention, which methods are defined and described in a number of patents and publications below, which are incorporated herein by reference. In PCT Patent Application No. WO 92/01813, Ruth and Driver disclosed a process for synthesizing circular single-stranded nucleic acids by hybridizing a linear polynucleotide to a complementary oligonucleotide and then ligating the linear polynucleotide. They further disclosed a process for generating multiple linear complements of the circular single-stranded nucleic acid template by extending a primer more than once around the circular template using a DNA polymerase. Japanese Patent Nos. JP4304900 and JP4262799 of Aono Toshiya et al. disclose detection of a target sequence by ligation of a linear single-stranded probe having target-complementary 3′- and 5′-end sequences which are adjacent when the linear probe is annealed to a target sequence in the sample, followed by either rolling circle replication or in vitro transcription of the circular single-stranded template. The inventors disclose that in vitro transcription is performed by first annealing to the circular single-stranded template a complementary nucleotide primer having an anti-promoter sequence in order to form a double-stranded promoter, and then transcribing the circular single-stranded template having the annealed anti-promoter primer with an RNA polymerase that has helicase-like activity, such as T7, T3 or SP6 RNA polymerase. In U.S. Pat. Nos. 6,344,329; 6,210,884; 6,183,960; 5,854,033; 6,329,150; 6,143,495; 6,316,229; and 6,287,824, Paul M. Lizardi also used rolling circle replication to amplify and detect nucleic acid sequences. In a series of articles and patents, Eric Kool and coworkers disclosed synthesis of DNA or RNA multimers, meaning multiple copies of an oligomer or oligonucleotide joined end to end (i.e., in tandem) by rolling circle replication or rolling circle transcription, respectively, of a circular DNA template molecule. Rolling circle replication uses a primer and a strand-displacing DNA polymerase, such as phi29 DNA polymerase. With respect to rolling circle transcription, it was shown that circular single-stranded DNA (ssDNA) molecules can be efficiently transcribed by phage and bacterial RNA polymerases (Prakash, G. and Kool, E., J. Am. Chem. Soc. 114: 3523-3527, 1992; Daubendiek, S. L. et al., J. Am. Chem. Soc. 117: 7818-7819, 1995; Liu, D. et al., J. Am. Chem. Soc. 118: 1587-1594, 1996; Daubendiek, S. L. and Kool, E. T., Nature Biotechnol., 15: 273-277, 1997; Diegelman, A. M. and Kool, E. T., Nucleic Acids Res., 26: 3235-3241, 1998; Diegelman, A. M. and Kool, E. T., Chem. Biol., 6: 569-576, 1999; Diegelman, A. M. et al., BioTechniques 25: 754-758, 1998; Frieden, M. et al., Angew. Chem. Int. Ed. Engl. 38: 3654-3657, 1999; Kool, E. T., Acc. Chem. Res., 31: 502-510, 1998; U.S. Pat. Nos. 5,426,180; 5,674,683; 5,714,320; 5,683,874; 5,872,105; 6,077,668; 6,096,880; and 6,368,802). Rolling circle transcription of these circular ssDNAs occurs in the absence of primers, in the absence of a canonical promoter sequence, and in the absence of any duplex DNA structure, and results in synthesis of linear multimeric complementary copies of the circle sequence up to thousands of nucleotides in length. Transcription of the linear precursor of the circular ssDNA template yielded only a small amount of RNA transcript product that was shorter than the template. Fire and Xu (U.S. Pat. No. 5,648,245; Fire, A. and Xu, S-Q, Proc. Natl. Acad. Sci. USA, 92: 4641-4645, 1995) also disclose methods for using rolling circle replication of small DNA circles to construct oligomer concatamers. Other researchers, including, but not limited to, Mahtani (U.S. Pat. No. 6,221,603), Rothberg et al. (U.S. Pat. No. 6,274,320), Dean et al. (Genome Res., 11: 1095-1099, 2001), Lasken et al. (U.S. Pat. No. 6,323,009), and Nilsson et al. (Nucleic Acids Res., 30(14): e66, 2002) disclose other methods and applications of rolling circle amplification. Pickering et al. (Nucleic Acids Res., 30(12): e60, 2002) discloses a ligation and rolling circle amplification method for homogeneous end-point detection of single nucleotide polymorphisms (SNPs).

As used herein, the terms “buffer” or “buffering agents” refer to materials that when added to a solution, cause the solution to resist changes in pH.

As used herein, the terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+ charge in solution (i.e., one more proton than electron).

As used herein, the term “divalent salt” refers to any salt in which a metal (e.g., Mg, Mn, Ca, or Sr) has a net 2+ charge in solution.

As used herein, the terms “chelator” or “chelating agent” refer to any materials having more than one atom with a lone pair of electrons that are available to bond to a metal ion.

As used herein, the term “solution” refers to an aqueous or non-aqueous mixture.

As used herein, the term “buffering solution” refers to a solution containing a buffering agent.

As used herein, the term “reaction buffer” refers to a buffering solution in which an enzymatic reaction is performed.

As used herein, the term “storage buffer” refers to a buffering solution in which an enzyme is stored.

In keeping with standard polypeptide nomenclature, J. Biol. Chem., 243:3557-3559, 1969, abbreviations for amino acid residues are as shown in the following Table of Correspondence.

TABLE OF CORRESPONDENCE

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

The phrase “mixed population” or “complex population” refers to any sample containing both desired and undesired nucleic acids. As a non-limiting example, a complex population of nucleic acids may be total genomic DNA, total cellular RNA or a combination thereof. Moreover, a complex population of nucleic acids may have been enriched for a given population, but include other undesirable populations. For example, a complex population of nucleic acids may be a sample that has been enriched for desired messenger RNA (mRNA) sequences but still includes some undesired ribosomal RNA sequences (rRNA).

The term “undetectable levels” of exonuclease activity with respect to a particular nucleic acid substrate refers to levels of digestion of a nucleic acid substrate that are not detectable within the assay or method in which an exonuclease is employed. Undetectable levels include the complete absence of digestion. Although the level of digestion of a nucleic acid of interest (i.e., a desired nucleic acid) can be undetectable in some embodiments, the invention is not limited to embodiments in which digestion of the nucleic acid of interest by a 5′-phosphate-dependent exonuclease of the invention is undetectable so long as a substantial amount of the undesired nucleic acid is digested and sufficient amount of the desired nucleic acid remains for the intended purpose, which can vary for different purposes. In preferred embodiments of the invention, a substantial amount of the undesired nucleic acid is digested by the 5′ exonuclease of the invention, which means that at least 50% (e.g., 60%, 70%, 80%, 90%, 95%, 98%, 99%) of the starting amount (i.e., the amount present prior to treatment with the exonuclease) of the undesired nucleic acid is digested.

The terms “sample” and “specimen” are used in their broadest sense and encompass samples or specimens obtained from any source including biological and environmental sources. As used herein, the term “sample” when used to refer to biological samples obtained from organisms, includes, but it not limited to fluids, solids, tissues, and gases. In preferred embodiments of this invention, biological samples include bodily fluids, isolated cells, fixed cells, cell lysates and the like. However, these examples are not to be construed as limiting the types of samples that find use with the present invention.

A “target nucleic acid” comprises at least one nucleic acid molecule or portion of at least one nucleic acid molecule, whether the molecule or molecules is or are DNA, RNA, or both DNA and RNA, and wherein each the molecule has, at least in part, a defined nucleotide sequence, which is referred to as the “target sequence.” A goal of an assay or method of the invention is to detect whether or not the target nucleic acid is present in a sample by means of methods that can detect the target sequence. This is frequently, but without limitation, achieved using a “nucleic acid probe” that is complementary to the target sequence (e.g., a probe that is present on an array or microarray together with other probes that are complementary to other nucleic acid sequences). The target nucleic acid may also have at least partial complementarity with other molecules used in an assay, such as, but not limited to, primers, splice template oligos, ligation splint oligos, capture probes or detection probes. The target nucleic acid may be single- or double-stranded and may be of any length. However, it must comprise a polynucleotide sequence of sufficient sequence specificity and length so as to be useful for its intended purpose. By way of example, but not of limitation, a target nucleic acid that is to be detected using a sequence-complementary detection probe must have a sequence of sufficient sequence specificity and length so as remain hybridized by the detection probe under assay hybridization conditions wherein sequences that are not target nucleic acids are not hybridized. A target nucleic acid having sufficient sequence specificity and length for an assay of the present invention may be identified, using methods known to those skilled in the art, by comparison and analysis of nucleic acid sequences known for a target and for other sequences which may be present in the sample. For example, sequences for nucleic acids of many viruses, bacteria, humans (e.g., for genes and messenger RNA), and many other biological organisms can be searched using public or private databases, and sequence comparisons, folded structures, and hybridization melting temperatures (i.e., Tₘ's) may be obtained using computer software known to those knowledgeable in the art. The term “source of target nucleic acid” refers to any sample that contains a naturally occurring target nucleic acid, RNA or DNA.

### DETAILED DESCRIPTION OF THE INVENTION

The present invention relates to compositions and methods employing 5′-phosphate-dependent nucleic acid exonucleases. In particular, the present invention provides kits and methods employing 5′-phosphate-dependent nucleic acid exonucleases for a variety of uses where it is desired to digest nucleic acid molecules having 5′-phosphate groups.

As defined herein, any exoribonuclease that selectively digests nucleic acid molecules having 5′-phosphate groups may be used in the methods and kits of the present invention. A 5′-to-3′ exoribonuclease of the present invention comprises any 5′-to-3′ exonuclease that has greater than 20-fold more 5′-to-3′ exonuclease activity for a single-stranded RNA substrate that has a 5′-monophosphorylated terminus than for the same RNA substrate that has a 5′-triphosphorylated or 5′-capped terminus. That is, a 5′ exonuclease of the present invention comprises any 5′-to′3′ exonuclease that has a relative activity in digesting a particular defined-sequence single-stranded RNA substrate with a 5′-triphosphate or a 5′-cap that is less than 5% of the activity of an RNA substrate having the same sequence but with a 5′-monophosphate. Without limitation, suitable methods that can be used for assaying activity and determining relative activity using RNA substrates with a 5′-triphosphate, a 5′-cap, or a 5′-monophosphate are described by Stevens and Poole (J. Biol. Chem., 270: 16063, 1995). Preferred embodiments employ Saccharomyces cerevisiae Xrn1p/5′ exoribonuclease 1 and/or functional variants and homologues thereof.

In some embodiments, the present invention provides a method of preparing a nucleic acid sample for analysis. It is often desirable to isolate, enrich, or increase the relative percentage of a particular population of sequences within a much larger population of sequences in order to limit analysis to sequences of interest and to reduce interference and unnecessary work that may be caused by the presence of undesirable sequences. The present invention provides a novel method wherein a complex sample or a mixed population of nucleic acids (e.g., containing both nucleic acids of interest and nucleic acids not of interest) is substantially depleted of undesired sequences (e.g., nucleic acids not of interest) and is thus enriched for a population of interest. For example, in preferred embodiments, the present invention provides a method of enriching a nucleic acid population of interest present in a complex sample by treating the sample with a 5′-phosphate-dependent nucleic acid exonuclease, thereby increasing the relative percentage of the nucleic acid population of interest in a given sample for further analysis. In preferred embodiments, the 5′-phosphate-dependent nucleic acid exonuclease digests greater than 50% (e.g., 60%, 70%, 80%, 90%, 95%, 97%, 99%, 99.5%, 99.9%) of the undesired sequence or sequences. Also, in preferred embodiments, the 5′-phosphate-dependent nucleic acid exonuclease digests less than 50%, (e.g., 40%, 30%, 20%, 15%, 10%, 5%, 4%, 3%, 2%, 1%) of the desired sequence or sequences, and most preferably, undetectable amounts of the desired sequence or sequences, but the invention also comprises embodiments in which more of the desired sequence or sequences is/are digested so long as the amount remaining can be used for the intended purpose and a substantial amount of the undesired sequence or sequences is/are digested.

For example, in some embodiments, methods of the present invention enrich for a nucleic acid population of interest (e.g., mRNA sequences) within a mixed population of nucleic acid sequences by targeting undesired sequences (e.g., 16S and 23S prokaryotic rRNA or 18S and 26S or 28S eukaryotic rRNA sequences comprising a 5′ phosphate) for digestion by a 5′-phosphate-dependent exonuclease and eliminating them from the mixed population. In some embodiments, the undesired sequences are degraded by the enzymatic activity of xrn-1 exoribonuclease (See, e.g., Example 1, Table 1).

The mixed population of nucleic acids may be any nucleic acid sample comprising both desired and undesired sequences. The population may include different DNA and/or RNA molecules. In some preferred embodiments, the mixed population is an RNA sample. In further preferred embodiments, the nucleic acid sample is RNA derived from a prokaryotic organism. In other preferred embodiments, the nucleic acid sample comprises RNA from a eukaryotic organism. In still other embodiments, the sample comprises RNA from multiple prokaryotic organisms, or from both prokaryotic and eukaryotic organisms, or from multiple eukaryotic organisms. The mixed population may be derived from a wide variety of sources including for example, tissue samples, blood, isolated cells or environmental samples such as water or soil. The mixed population may be derived from any organism including both eukaryotes and prokaryotes such as human, rat, mouse, Escherichia coli (E. coli), Bacillus subtilis (B. subtilis), Pseudomonas aeruginosa, etc. Methods of deriving nucleic acid samples from eukaryotic and prokaryotic organisms are well known to those of skill in the art (See, e.g., Chapter 4, “Current Protocols in Molecular Biology,” Ausubel et al., eds (1997 supplement) Johan Wilen & Sons, Inc. and Chapter 7, Sambrook, Fritsch, Maniatis “Molecular Cloning: A Laboratory Manual” (1989) Cold Spring Harbor Press, etc.).

The population of interest may comprise a subset of the mixed population. The population of interest may include RNA and/or DNA. The population of interest may, for example, be a particular type of RNA. In a preferred embodiment the population of interest is mRNA. The population of interest may comprise any sequence or mixture of sequences and the sequence or mixture of sequences need not be known. The population of interest may be chosen on a variety of bases, including by sequence, function (e.g. messenger RNA (mRNA), ribosomal RNA (rRNA), transfer RNA (tRNA), etc.), or a combination thereof. In some embodiments, the population of interest is provided in a sample that has been depleted of 5S rRNA and/or tRNA. Such a mixture may be generated by salt precipitation using LiCl. The larger rRNAs and mRNAs are precipitated, but the tRNAs and 5S rRNAs remain in solution and can be removed. In some embodiments, the tRNAs and 5S rRNAs are removed by LiCl precipitation after a sample is treated with exoribonuclease. Kits for conducting such reactions are provided by the present invention (e.g., kits containing LiCl and exoribonuclease). Other methods for isolating subsets of molecules (e.g., PEG precipitation, etc.) may also be employed in the kits and methods of the present invention.

The sequences that are targeted for digestion may comprise undesired sequences in the mixed population. The sequences that are targeted for digestion may comprise any sequence so long as they are distinguishable from the population of interest (e.g., the sequences that are targeted for digestion possesses an accessible 5′ phosphate whereas the population of interest does not). Targeted double-stranded DNA (dsDNA) or RNA (dsRNA) or single-stranded DNA (ssDNA) or RNA (ssRNA) that self-anneals to form double-stranded regions can be denatured by various treatments known in the art, such as, but not limited to, treatment with heat or addition of compositions that affect the melting temperature of the targeted polynucleotide, such as, but not limited to betaine. In preferred embodiments, the sequences that are targeted for digestion are RNAs including rRNA (e.g., 16S and 23S prokaryotic rRNA, or 18S and 26S or 28S eukaryotic rRNA). In some embodiments, it may not be necessary to remove substantially all the undesired sequences from the mixed population. In these embodiments it is acceptable to remove only enough of the undesired sequences such that the undesired sequences do not interfere with analysis of the population of interest.

In preferred embodiments, any non-targeted undesirable sequences represent only a small proportion of the mixed population or are readily removed using other methods known in the art. These non-targeted undesirable sequences may include a variety of other nucleic acids such as DNAs, rRNAs, mRNAs or tRNAs. For the sake of simplicity, the presence of non-targeted RNAs will not be discussed throughout the remainder of the application; however, the possibility of their presence is contemplated by the scope of the presently claimed invention, unless described otherwise.

A particular example of the presently claimed invention provides a method of isolating or enriching for mRNAs within a mixed population of RNAs by specifically removing rRNAs >300 nucleotides that possess an accessible 5′ phosphate. A mixed population of RNAs may include, for example, mRNAs, tRNAs and rRNAs.

In some embodiments, methods of the present invention are used in combination with methods that expose a 5′ phosphate on an undesired nucleic acid sequence (e.g., a method that removes a 5′-triphosphate or a 5′ cap on an undesired nucleic acid sequence using an agent, such as, but not limited to, tobacco acid pyrophosphatase).

In other embodiments, methods of the present invention are used in combination with methods that result in addition of a 5′-phosphate to a nucleic acid molecule of interest (e.g., a method for 5′-phosphorylation using a polynucleotide kinase, such as, but not limited to, T4 polynucleotide kinase).

In other embodiments, methods of the present invention are used in combination with methods that result in addition of a cap to the 5′-terminus of a nucleic acid sequence (e.g., a method for 5′-capping of an mRNA molecule having a 5′-triphosphate using a guanyltransferase, such as, but not limited to, Vaccinia virus capping enzyme).

In some preferred embodiments, the sample containing the nucleic acids of interest (e.g., mRNA sequences) and the undesired nucleic acids (e.g., rRNA sequences) is partially purified prior to treatment using a composition or method of the present invention. By way of example, but not of limitation, total RNA can be purified from a sample using methods known in the art, including, for example, commercially available purification kits such as the MasterPure™ RNA Purification Kit (EPICENTRE Technologies, Madison, Wis.) or the RNeasy Kit (Qiagen, Valencia, Calif.) prior to treatment with an exoribonuclease according to the present invention.

In preferred embodiments, once the undesired nucleic acids (e.g., rRNA sequences) are eliminated, the nucleic acids of interest (e.g., mRNA sequences) are further purified using methods known in the art, including, for example, commercially available purification kits such as the MasterPure™ Complete DNA and RNA Purification Kit (EPICENTRE Technologies, Madison, Wis.) or the RNeasy Kit (Qiagen, Valencia, Calif.).

In some embodiments, the presently claimed invention provides a method of differentiating between nucleic acid sequences. For example, the present invention provides a method of distinguishing nucleic acid sequences that comprise an accessible 5′ phosphate (e.g., that are targeted for digestion by a 5′-phosphate-dependent exonuclease) from nucleic acid sequences that do not possess an accessible 5′ phosphate (e.g., that are not targeted for digestion by xrn-1). Thus, the present invention provides diagnostic methods. A wide variety of diagnostic methods are provided by the invention. Any method that utilizes an exoribonuclease to distinguish between nucleic acid molecules having and lacking 5′ phosphate groups is encompassed by the present invention. For example, the exoribonucleases may be used to identify nucleic acid molecules that have been digested by restriction enzyme—generating a 5′ phosphate—as compared to undigested nucleic acid molecules lacking a 5′ phosphate.

In some embodiments, the method of differentiating between nucleic acids is used in combination with known diagnostic methods. For example, in some embodiments, methods of using exoribunucleases of the present invention are combined with restriction enzyme digestion used in restriction fragment length polymorphism (RFLP) analysis or other diagnostic methods.

In some embodiments, the enzymatic activity of the exoribonuclease of the present invention is controlled by altering the concentration of a divalent cation (e.g., Mg²⁺) present in a reaction solution. In some embodiments, the enzymatic activity of exoribonuclease of the present invention is controlled by addition of a chelating agent.

### EXAMPLES

The following examples serve to illustrate certain preferred embodiments and aspects of the present invention and are not to be construed as limiting the scope thereof.

### Example 1

Activity of Exoribonuclease I

This Example describes the use of Exoribonuclease I in enriching for specific RNA substrates in the presence of other undesired nucleic acids.

A. Materials and Methods

Enzyme

Exoribonuclease I (xrn-1) (Stevens, Biochem. Biophys. Res. Commun. 81:656, 1978); Stevens, Biochem. Biophys. Res. Commun. 86:1126, 1979); Stevens, J. Biol. Chem 255:3080, 1980); and Stevens and Maupin, Nucleic Acids Res. 15:695, 1987) was purified from a recombinant source using methods similar to those described in the art. The enzyme was stored in 50% (v/v) glycerol containing, 0.05 M Tris-HCl (pH 7.5), 100 mM NaCl, 0.1 mM EDTA, 1 mM DTT, and 0.1% Triton X-100.

One unit of exoribonuclease I is the amount of enzyme that converts 1 μg of E. coli ribosomal RNA (comprising 16S and 23S rRNA) to acid-soluble form in 60 minutes at 30° C. under standard reaction assay conditions. Exoribonuclease I used in the experiments described herein is free of other detectable contaminating ribonucleases, as well as free from detectable contaminating exo- and endodeoxyribonucleases (although the enzyme itself has DNase activity).

Reaction Conditions

All experiments were performed in a reaction buffer containing 50 mM Tris-HCl (pH 8.0), 2.0 mM MgCl₂, and 100 mM NaCl.

B. Results.

Capped RNA is Resistant to xrn-1 Degradation

A 75-nucleotide, capped transcript was made with AmpliCap™ SP6 High Yield Message Maker Kit from EPICENTRE (Madison, Wis.). Samples containing this capped RNA were treated with tobacco acid pyrophosphatase (TAP; EPICENTRE) to remove the cap (or “de-cap”) the RNA and/or with xrn-I, as indicated in Table 1, and analyzed by polyacrylamide gel electrophoresis. The results, shown in Table 1, demonstrated that the 5′-capped RNA was resistant to digestion (or “degradation”) by xrn-1. De-capping the RNA with TAP resulted in degradation of the RNA by xrn-1 exonuclease.

TABLE 1

| TAP Treated | Xrn-1 Treated | Degradation |
|---|---|---|
| − | − | − |
| − | + | − |
| + | − | − |
| + | + | + |

Degradation of Ribosomal RNA

A preparation of increasing amounts of tobacco RNA isolated from tobacco plants was treated with xrn-1 exonuclease and analyzed by agarose gel electrophoresis. Minimal RNA was visible in the lanes containing RNA treated with xrn-1. These results showed that the xrn-1 exonuclease was able to digest abundant ribosomal RNA.

The experiment was next repeated with a mixture of the tobacco RNA and a 1.4-kB capped mRNA transcript prepared using an AmpliCap™ T7 High Yield Message Maker Kit (EPICENTRE), which was added to the preparation of tobacco RNA, and with the 1.4-kB transcript alone. Results of the reactions were analyzed by agarose gel electrophoresis. The capped mRNA was not degraded by xrn-1. In the lanes containing both the tobacco RNA and the capped mRNA treated with xrn-1, the ribosomal RNA bands corresponding to the tobacco RNA were degraded, but those corresponding to the capped mRNA were not degraded. The present invention is not limited to a particular mechanism. Indeed, an understanding of the mechanism is not necessary to practice the present invention. Nonetheless, it is contemplated that the tobacco mRNA remains undegraded (see above) but is not visible due to its low concentration.

The xrn-1-treated tobacco RNA was concentrated approximately 50× by ethanol precipitation and analyzed on an agarose gel. The results indicated that the bulk of the RNA was degraded, but that a small amount of xrn-1-resistant RNA was present that appeared as a heterodisperse size range that could be observed on an agarose gel following ethanol concentration.

5′ pppG RNAs are Resistant to Degradation

A T7 control DNA template was used to synthesize a sample comprising a 1.4-kB RNA transcript using either: a) standard rNTPS without a cap; b) a capping rNTP mixture containing rNTPs and m⁷ GpppG; or c) a non-capping non-pppG 5′ end mix containing rNTPs and an rGMP (which can be incorporated at the 5′-end of a transcript during in vitro transcription instead of a 5′-triphosphorylated G). Each transcript preparation was treated with xrn-1, and then xrn-1-treated and xrn-1-untreated samples were analyzed by agarose gel electrophoresis. The 5′ pppG RNA was resistant to degradation by xrn-1. The RNA transcript that lacked a 5′-triphosphate or a cap structure was degraded by xrn-1. The RNA with rGMP in the transcription reaction was partially sensitive to degradation by xrn-1, presumably because some of the transcripts had a 5′-monophosphate due to incorporation of the GMP during in vitro transcription. These results show that xrn-1 can be used to enrich for uncapped but 5′-triphosphorylated RNA (e.g., bacterial mRNA).

xrn-1 Degradation of E. coli RNA

E. coli total RNA was treated with xrn-1 exonuclease. Following xrn-1 treatment, a portion of the RNA preparation was concentrated approximately 20× by ethanol precipitation and analyzed by agarose gel electrophoresis. The majority of the RNA, including the E. coli 16S and 23S rRNA, was degraded. Following ethanol precipitation to concentrate the RNA remaining in the xrn-1-treated solution, a heterodisperse size range of undegraded RNA was observed on the agarose gel. Specific mRNAs for different genes were detected by RT-PCR of the xrn-1-treated material.

### Example 2

Enrichment of Eukaryotic mRNA from Eukaryotic Total RNA for Use in cDNA Synthesis, RNA Amplification and Preparation of Labeled Target RNA for Gene Expression Analysis Using DNA Microarrays.

Sample Source and Nucleic Acid Samples

Cells from a mouse stem cell line that had been transformed with a gene linked to an inducible promoter were treated with the inducing substance and then grown in culture for either 6 days or 12 days following induction. Total RNA was then isolated from each 6-day and 12-day culture using methods known in the art, resulting in samples that were designated as the “6-day sample” and the “12-day sample,” respectively. Each RNA sample had prominent 18S and 28S rRNA peaks when they were analyzed using an Agilent 2100 bioanalyzer.

xrn-1 Treatment of 6-Day and 12-Day RNA Samples

The 6-day and 12-day RNA samples were each divided into two aliquots. The first aliquot was treated with xrn-1 exonuclease and the second aliquot was not treated with xrn-1 exonuclease. No 18S or 28S rRNA peaks are detectable using the Agilent 2100 bioanalyzer after the total RNA is treated with the xrn-1 exonuclease using the same standard protocol.

cDNA Synthesis and RNA Amplification of xrn-1-Treated and xrn-1-Untreated 6-Day and 12-Day RNA Samples

Two hundred picograms of total RNA from the xrn-1-untreated aliquots of 6-day and 12-day RNA samples, or a volume of the xrn-1-treated aliquots equivalent to the volume of 200 pg of total RNA in the xrn-1-untreated aliquots, were each used for cDNA and aminoallyl-antisense RNA (“AA-aRNA”) synthesis using the TargetAmp™ 2-Round Aminoallyl-aRNA Amplification Kit 1.0 (EPICENTRE) and SuperScript™_III and SuperScript™ II reverse trancriptases (INVITROGEN) for cDNA synthesis in the first and second rounds of amplification, respectively, according to the instructions provided in the TargetAmp 1.0 kit; the TargetAmp 1.0 kit makes AA-aRNA corresponding to mRNA in the sample using an Eberwine-type aRNA amplification process (Van Gelder, R. N., et al., Proc. Natl. Acad. Sci. USA, 87: 1663, 1990). Sufficient amounts of AA-aRNA were obtained from amplification reactions for all four types of samples for use in preparing labeled target RNA for hybridization to Affymetrix mouse whole genome GeneChip®s.

Microarray Analysis of Amplified aRNA from mRNA in xrn-1-Treated and xrn-1-Untreated 6-Day and 12-Day RNA Samples

Prior to hybridization to an Affymetrix GeneChip, each AA-aRNA was biotinylated using Biotin-X-X-NHS (EPICENTRE) according to the protocol of the supplier. The biotinylated a-RNA was then fragmented, and hybridized to mouse genome GeneChip arrays (MG 430 2.0, Affymetrix) in triplicates. Hybridization, staining, and washing were conducted as specified by Affymetrix. Data were analyzed using GeneSpring software (Agilent Technologies).

Microarray Results Using Labeled Target aRNA Prepared from xrn-1-Treated and xrn-1-Untreated 6-Day and 12-Day RNA Samples

The “% present calls” were 49-51% from all GeneChips hybridized to labeled target aRNA prepared from xrn-1-treated or xrn-1-untreated 12-day RNA samples, and ˜43-48% for both 6-day samples (excluding one xrn-1-untreated replicate that had a “% present call” of only 29.2%). The concordance of present and absent calls between the xrn-1-treated and xrn-1-untreated 12-day samples was 73.6%. This concordance was close to the average concordance (82.6%) obtained from pairwise comparisons of three replicate slides hybridized with biotinylated aRNA samples obtained from untreated RNA. Thus, the high concordance indicates good preservation of messenger RNA in the xrn-1 treated samples.

### Example 3

Preservation of Relative mRNA Abundance Levels after xrn-1 Exonuclease Treatment.

Two micrograms of human reference RNA and human skeletal muscle RNA were treated with 1 U of xrn-1 exonuclease for 1 hour at 30° C. The RNA was extracted and concentrated by ethanol precipitation and the entire sample was used in a 40 μl reverse transcription reaction using MMLV reverse transcriptase (EPICENTRE) with random nonamer primers at 37° C. for 1 hour. qPCR was performed using TAQURATE GREEN Real-time PCR MasterMix (EPICENTRE) and optimized concentrations of target-specific primers. The abundance of beta-2-microglobulin (B2M) in the samples was used for expression level normalization. The expression levels of six different target messages were analyzed. Duplicate cDNA and qPCR reactions were preformed, averaged, and normalized for each comparison. Simultaneous analysis was performed with normalization and test primers, and non-template controls were included. The difference in threshold cycles between a target message in one RNA sample (in relation to the normalizer) and that of the other RNA sample (in relation to the normalizer) was determined. The results showed that xrn-1 exonuclease maintained mRNA abundance levels.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

### Amendments to the Specification

Please insert the attached Sequence Listing into the specification after the abstract.

We claim:

1. A method for enriching for an RNA having a 5′-triphosphate or a 5′-cap in a biological sample comprising prokaryotic RNA, eukaryotic RNA or both prokaryotic and eukaryotic RNA and at least one undesired nucleic acid, the method comprising treating the sample with purified 5′ exoribonuclease under conditions in which the 5′ exoribonuclease is active and for sufficient time so that the undesired nucleic acid is digested and the sample is enriched for RNA having a 5′-triphosphate or a 5′-cap.

2. The method of claim 1, wherein the RNA having a 5′-triphosphate or a 5′-cap is selected from the group consisting of: (i) prokaryotic mRNA; (ii) eukaryotic mRNA, including polyadenylated and non-polyadenylated eukaryotic mRNA; (iii) a mixture of both prokaryotic and eukaryotic mRNA; (iv) eukaryotic snRNA; (v) eukaryotic pre-micro RNA; and (vi) prokaryotic or eukaryotic primary RNA transcripts.

3. The method of claim 1, wherein the RNA having a 5′-triphosphate or a 5′-cap comprises eukaryotic mRNA or both prokaryotic and eukaryotic mRNA, and wherein the method additionally comprises binding the RNA to an oligo(dT) or oligo(dU) resin, membrane or other surface to which oligo(dT) or oligo(dU) is attached and eluting the bound RNA so as to obtain a solution containing polyadenylated eukaryotic mRNA.

4. The method of claim 1 wherein the RNA having a 5′-triphosphate or a 5′-cap comprises prokaryotic mRNA, eukaryotic mRNA or both prokaryotic and eukaryotic mRNA and the undesired nucleic acid comprises prokaryotic rRNA, eukaryotic rRNA or both prokaryotic and eukaryotic rRNA of a size having a svedburg unit greater than about 10S.

5. The method of claim 4 wherein the rRNA is selected from the group consisting of prokaryotic rRNA having a svedburg unit of about 16S or 23S and eukaryotic rRNA having a svedburg unit of about 18S, 26S or 28S.

6. The method of claim 1 wherein the RNA having a 5′-triphosphate or a 5′-cap comprises prokaryotic mRNA, eukaryotic mRNA, or both prokaryotic and eukaryotic mRNA, and wherein the method additionally comprises precipitation of said mRNA having a 5′-triphosphate or a 5′-cap with a solution of LiCl and ethanol at concentrations and under conditions in which tRNA and 5S rRNA are not precipitated.

7. The method of claim 6 wherein the RNA having a 5′-triphosphate or a 5′-cap comprises prokaryotic mRNA, eukaryotic mRNA, or both prokaryotic and eukaryotic mRNA and the undesired nucleic acid comprises prokaryotic rRNA, eukaryotic rRNA or both prokaryotic and eukaryotic rRNA of a size having a svedburg unit greater than about 10S.

8. The method of claim 7 wherein the rRNA is selected from the group consisting of prokaryotic rRNA having a svedburg unit of about 16S or 23S and eukaryotic rRNA having a svedburg unit of about 18S, 26S or 28S.

9. The method of claim 1, wherein the biological sample is treated with a polynucleotide kinase to phosphorylate the RNA having 5′-hydroxyl groups prior to treating the sample with purified 5′ exoribonuclease.

10. The method of claim 1, wherein the method additionally comprises: (i) contacting the sample enriched for RNA having a 5′-triphosphate or a 5′-cap with a poly(A) polymerase under conditions so that polyadenylated RNA is obtained; and (ii) contacting the polyadenylated RNA with an oligo(dT)-containing primer and an RNA-dependent DNA polymerase under conditions that cDNA complementary to the RNA is obtained.

11. The method of claim 9, wherein the biological sample is treated with a polynucleotide kinase to phosphorylate the RNA having 5′-hydroxyl groups prior to treating the sample with purified 5′ exoribonuclease.

12. The method of claim 10, wherein the oligo(dT)-containing primer comprises a T7-type RNA polymerase promoter selected from the group consisting of T7 RNA polymerase, T3 RNA polymerase, and SP6 RNA polymerase.

13. The method of claim 1, wherein the biological sample comprises eukaryotic RNA, and wherein the method additionally comprises contacting the sample enriched for RNA having a 5′-triphosphate or a 5′-cap with an oligo(dT)-containing primer and an RNA-dependent DNA polymerase under conditions that cDNA complementary to the RNA is obtained.

14. The method of claim 13, wherein the biological sample is treated with a polynucleotide kinase to phosphorylate the RNA having 5′-hydroxyl groups prior to treating the sample with purified 5′ exoribonuclease.

15. The method of claim 13, wherein the oligo(dT)-containing primer comprises a T7-type RNA polymerase promoter selected from the group consisting of T7 RNA polymerase, T3 RNA polymerase, and SP6 RNA polymerase.

16. The method of claim 15, wherein the method additionally comprises contacting the cDNA complementary to the RNA with one or more DNA or RNA primers and a DNA polymerase and incubating under conditions so as to obtain double-stranded cDNA having a functional promoter for the T7-type RNA polymerase and synthesizing RNA therefrom.

17. The method of claim 13, wherein the method additionally comprises contacting the cDNA complementary to the RNA with an oligonucleotide sequence tag template and a DNA polymerase and incubating under conditions so as to obtain cDNA having a sequence tag on it the 3′-end.

18. The method of claim 17, additionally comprising contacting the cDNA having the sequence tag on its 3′-end with a DNA polymerase and an oligonucleotide primer, wherein the either the sequence tag or the oligonucleotide primer, or the combination of both the sequence tag and the oligonucleotide primer encodes the complete sequence of a T7-type RNA polymerase promoter, and incubating under conditions wherein double-stranded cDNA having a functional T7-type RNA polymerase promoter is obtained.

19. The method of claim 18, wherein the method additionally comprises contacting the cDNA with the T7-type RNA polymerase under conditions so as to synthesize RNA using said T7-type RNA polymerase promoter.

20. The method of claim 1, wherein the 5′ exoribonuclease is a wild-type or recombinant Saccharomyces cerevisiae Xrn1p/5′ exoribonuclease 1.

21. A kit for enriching for mRNA having a 5′-triphosphate or a 5′-cap in a biological sample comprising prokaryotic mRNA, eukaryotic mRNA or both prokaryotic and eukaryotic mRNA and at least one undesired nucleic acid, the kit comprising: (i) a solution of purified and stabilized 5′ exoribonuclease; and (ii) a concentrated solution of a reaction buffer for providing a 1× reaction buffer in which the 5′ exoribonuclease is active.

22. The kit of claim 21, additionally comprising one or more components selected from the group consisting of: (i) a negative control comprising an RNA having a 5′-triphosphate or a 5′-cap; (ii) a positive control comprising an RNA having a 5′-monophosphate; (iii) a solution of LiCl; (iv) a polynucleotide kinase; and (v) a poly(A) polymerase.

23. The kit of claim 21, additionally comprising an RNA-dependent DNA polymerase (reverse transcriptase).

24. The kit of claim 21, additionally comprising one or more components selected from the group consisting of: (i) a poly(A) polymerase; (ii) an oligo(dT)-containing primer; and (iii) an RNA-dependent DNA polymerase.

25. The kit of claim 21, additionally comprising one or more components selected from the group consisting of: (i) a polynucleotide kinase; (ii) an oligo(dT)-containing primer; and (iii) an RNA-dependent DNA-polymerase.

26. The kit of claim 21, additionally comprising one or more primers selected from the group consisting of (i) an oligo(dT)-containing primer; and (ii) a primer that is complementary to a specific nucleic acid sequence.

27. The kit of claim 26, wherein said primer additionally comprises a sense or an antisense sequence of a double-stranded promoter for a T7-type RNA polymerase selected from the group consisting of T7 RNA polymerase, T3 RNA polymerase, and SP6 RNA polymerase, and wherein the kit additionally comprises the RNA polymerase that can transcribe RNA using said promoter.

28. The kit of claim 26, additionally comprising: (i) a oligonucleotide sequence tag template; and (ii) a DNA polymerase selected from the group consisting of a DNA-dependent DNA polymerase and an RNA-dependent DNA polymerase that can extend the 3′-end of a nucleic acid that is annealed to the oligonucleotide sequence tag template.

29. The kit of claim 28, additionally comprising: (i) an oligonucleotide that is complementary to the oligonucleotide sequence tag template; and (ii) a T7-type RNA polymerase selected from the group consisting of T7 RNA polymerase, T3 RNA polymerase, and SP6 RNA polymerase; wherein the oligonucleotide sequence tag template and the oligonucleotide that is complementary to the oligonucleotide sequence tag template together encode the complete sequence of a promoter that can be used for transcription by said T7-type RNA polymerase.

30. The kit of claim 21, additionally comprising one or more components selected from the group consisting of: (i) a negative control comprising an RNA having a 5′-triphosphate or a 5′-cap; (ii) a negative control comprising an RNA or a DNA having a 5′-hydroxyl group; (iii) a positive control comprising an RNA having a 5′-monophosphate; (iv) a solution of LiCl; (v) a ribonuclease H enzyme; and (vi) a polynucleotide kinase.

31. The kit of claim 30, additionally comprising one or more components selected from the group consisting of: (i) a poly(A) polymerase; (ii) an oligo(dT)-containing primer; and (iii) an RNA-dependent DNA polymerase.
(for prokaryotic mRNA)

32. The kit of claim 30, additionally comprising an RNA-dependent DNA polymerase and one or more primers selected from the group consisting of (i) a random primer, such as a random hexamer primer; (ii) an oligo(dT)-containing primer; and (iii) a primer that is complementary to a specific nucleic acid sequence.

33. The kit of claim 30, additionally comprising: (i) an oligonucleotide sequence tag template for adding a tag sequence to the 3′-end of mRNA having a 5′-triphosphate or a 5′-cap; and (ii) a DNA polymerase selected from the group consisting of a DNA-dependent DNA polymerase and an RNA-dependent DNA polymerase that can extend the 3′-end of the mRNA using the oligonucleotide sequence tag template annealed to the mRNA as a template.

34. The kit of claim 21, additionally comprising: (i) an oligonucleotide sequence tag template for adding a tag sequence to the 3′-end of mRNA having a 5′-triphosphate or a 5′-cap; and (ii) a DNA polymerase selected from the group consisting of a DNA-dependent DNA polymerase and an RNA-dependent DNA polymerase that can extend the 3′-end of the mRNA using the oligonucleotide sequence tag template annealed to the mRNA as a template.

35. The kit of claim 34, wherein the DNA polymerase is an RNA-dependent DNA polymerase, or if a DNA-dependent DNA polymerase is used for adding the tag sequence to the 3′-end of the mRNA, wherein the kit additionally contains an RNA-dependent DNA polymerase that can synthesize cDNA from a primer using the mRNA as a template.

36. The kit of claim 34, wherein said oligonucleotide sequence tag template additionally encodes either a sense or an antisense strand of a double-stranded promoter for a T7-type RNA polymerase selected from the group consisting of T7 RNA polymerase, T3 RNA polymerase, and SP6 RNA polymerase, and wherein the kit additionally comprises the RNA polymerase that can transcribe RNA using said promoter.

37. The kit of claim 21, additionally comprising: (i) a poly(A) polymerase; (ii) an oligo(dT)-containing primer; and (iii) an RNA-dependent DNA polymerase.

38. The kit of claim 37, additionally comprising: (i) an oligonucleotide sequence tag template; (ii) an oligonucleotide that is complementary to the oligonucleotide sequence tag template; wherein the oligonucleotide sequence tag template and the oligonucleotide complementary to the oligonucleotide sequence tag template together encode the complete sequence of a promoter for a T7-type RNA polymerase.

39. The kit of claim 37, additionally comprising a T7-type RNA polymerase selected from the group consisting of a T7 RNA polymerase, a T3 RNA polymerase, and an SP6 RNA polymerase, wherein the T7-type RNA polymerase can use the promoter encoded by the oligonucleotide sequence tag template and the oligonucleotide complementary to the oligonucleotide sequence tag template.

40. A kit for removing intact prokaryotic or eukaryotic ribosomal RNA (rRNA) of a size having a svedburg unit greater than about 10S from a biological sample, the kit comprising: (i) a solution of purified and stabilized 5′ exoribonuclease; (ii) a concentrated solution of a reaction buffer for providing a 1× reaction buffer in which the exoribonuclease is active; and (iii) a positive control rRNA, selected from the group consisting of a 16S and a 23S prokaryotic rRNA, and an 18S, a 26S and a 28S eukaryotic rRNA.

41. The kit of claim 40, wherein the 5′ exoribonuclease is a wild-type or recombinant Saccharomyces cerevisiae Xrn1p/5′ exoribonuclease 1.


SEQUENCE LISTING (2 sequences)

SEQ ID NO: 1 - protein, 1528 aa, Saccharomyces cerevisiae
MGIPKFFRYISERWPMILQLIEGTQIPEFDNLYLDMNSILHNCTHGNDDDVTKRLTEEEVFAKICTYIDHLFQTIKPKKIFYMAIDGVAPRAKMNQQRARRFRTAMDAEKALKKAIENGDEIPKGEPFDSNSITPGTEFMAKLTKNLQYFIHDKISNDSKWREVQIIFSGHEVPGEGEHKIMNFIRHLKSQKDFNQNTRHCIYGLDADLIMLGLSTHGPHFALLREEVTFGRRNSEKKSLEHQNFYLLHLSLLREYMELEFKEIADEMQFEYNFERILDDFILVMFVIGNDFLPNLPDLHLNKGAFPVLLQTFKEALLHTDGYINEHGKINLKRLGVWLNYLSQFELLNFEKDDIDVEWFNKQLENISLEGERKRQRVGKKLLVKQQKKLIGSIKPWLMEQLQEKLSPDLPDEEIPTLELPKDLDMKDHLEFLKEFAFDLGLFITHSKSKGSYSLKMDLDSINPDETEEEFQNRVNSIRKTIKKYQNAIIVEDKEELETEKTIYNERFERWKHEYYHDKLKFTTDSEEKVRDLAKDYVEGLQWVLYYYYRGCPSWSWYYPHHYAPRISDLAKGLDQDIEFDLSKPFTPFQQLMAVLPERSKNLIPPAFRPLMYDEQSPIHDFYPAEVQLDKNGKTADWEAVVLISFVDEKRLIEAMQPYLRKLSPEEKTRNQFGKDLIYSFNPQVDNLYKSPLGGIFSDIEHNHCVEKEYITIPLDSSEIRYGLLPNAKLGAEMLAGFPTLLSLPFTSSLEYNETMVFQQPSKQQSMVLQITDIYKTNNVTLEDFSKRHLNKVIYTRWPYLRESKLVSLTDGKTIYEYQESNDKKKFGFITKPAETQDKKLFNSLKNSMLRMYAKQKAVKIGPMEAIATVFPVTGLVRDSDGGYIKTFSPTPDYYPLQLVVESVVNEDERYKERGPIPIEEEFPLNSKVIFLGDYAYGGETTIDGYSSDRRLKITVEKKFLDSEPTIGKERLQMDHQAVKYYPSYIVSKNMHLHPLFLSKITSKFMITDATGKHINVGIPVKFEARHQKVLGYARRNPRGWEYSNLTLNLLKEYRQTFPDFFFRLSKVGNDIPVLEDLFPDTSTKDAMNLLDGIKQWLKYVSSKFIAVSLESDSLTKTSIAAVEDHIMKYAANIEGHERKQLAKVPREAVLNPRSSFALLRSQKFDLGDRVVYIQDSGKVPIFSKGTVVGYTTLSSSLSIQVLFDHEIVAGNNFGGRLRTNRGLGLDASFLLNITNRQFIYHSKASKKALEKKKQSNNRNNNTKTAHKTPSKQQSEEKLRKERAHDLLNFIKKDTNEKNSESVDNKSMGSQKDSKPAKKVLLKRPAQKSSENVQVDLANFEKAPLDNPTVAGSIFNAVANQYSDGIGSNLNIPTPPHPMNVVGGPIPGANDVADVGLPYNIPPGFMTHPNGLHPLHPHQMPYPNMNGMSIPPPAPHGFGQPISFPPPPPMTNVSDQGSRIVVNEKESQDLKKFINGKQHSNGSTIGGETKNSRKGEIKPSSGTNSTECQSPKSQSNAADRDNKKDEST

SEQ ID NO: 2 - DNA, 4580 bp, Saccharomyces cerevisiae
atgggtattccaaaatttttcaggtacatctcagaaagatggcccatgattttacagcttattgagggaacacagattcctgagtttgataacttatacctggatatgaattcgattttacataattgtacgcatggtaacgacgatgatgtaaccaagcgattaactgaagaagaggtttttgcaaaaatctgtacgtatatcgatcacctttttcaaacaatcaagcccaagaagattttctacatggctattgatggtgtggcccctcgtgccaagatgaatcaacaaagagctcgtagattcagaaccgctatggatgcagaaaaagccttgaagaaggctattgagaatggtgacgagattcctaagggtgagccatttgattcgaattctattactccaggtacggagtttatggccaaattgaccaaaaacttacaatattttattcacgacaagatttctaacgattccaaatggagggaagtgcaaatcatattttctggccatgaagttccaggtgaaggtgaacacaagatcatgaactttataaggcatttaaaatcccaaaaggatttcaaccagaatacgagacattgtatttacggtcttgacgcagatttgattatgctgggtttgtctactcatgggccacattttgcgttattgagagaagaagtgacatttggtagaagaaatagtgaaaaaaaatcgcttgaacatcaaaatttctacttattacatctttctttattaagagaatacatggagttggaattcaaagaaattgccgatgaaatgcaatttgaatacaattttgaacgtattttggatgattttattcttgtcatgttcgtcattggtaatgatttcttgcccaatttgccagatttgcaccttaacaaaggagcatttcccgttttgttacaaacgttcaaagaagctcttttacatactgatggctacattaatgaacatggtaaaataaatttaaagagattaggtgtctggttaaattatctgtctcaatttgagttattaaatttcgaaaaggatgatatagacgttgagtggttcaacaagcaattagagaatatttctttggagggtgaaaggaaaagacagagggttggtaaaaaattactggtaaaacaacagaagaaattaattggaagtataaaaccatggttgatggagcaattacaggaaaaattatcgcctgatttaccagatgaagaaattccaactttagagttacctaaggacttagacatgaaagatcatttagaatttttaaaagaattcgcttttgatttgggtctttttataacgcattccaaatccaaaggtagttattcgctaaaaatggatcttgattctattaatcctgatgaaacagaagaagaatttcaaaatcgtgttaattctatcaggaaaacaataaaaaaatatcaaaatgctatcatcgtggaggacaaagaagaattggaaactgaaaaaacgatttataatgaaaggtttgaacgttggaagcatgagtattatcacgacaagttaaaatttacgacagacagtgaagaaaaagtgagagatcttgctaaagactacgttgaaggtttacaatgggttctatattattattatagaggatgtccatcttggtcgtggtactatccgcaccattatgcaccaagaatctccgacttagccaagggtttagatcaagacattgaatttgatttgagcaaaccatttactccattccagcaactaatggcagttttaccggaaaggtccaaaaatctgatacctcccgcctttaggccattaatgtacgatgaacagtcgccaatccacgatttctatcccgctgaggttcaacttgataaaaacggcaagacagctgattgggaagctgtggttttgatatcgtttgtagatgaaaaaaggttgattgaggctatgcaaccttatttgcgcaagttatcacctgaagaaaaaacgagaaatcaatttggcaaggacttgatatattcctttaatcctcaagttgataacctttataagagtccgttgggcggcattttttctgatattgaacacaatcattgtgtcgaaaaagagtacatcaccatcccattggacagctccgagattcggtatggtttattacctaatgctaaactcggtgccgaaatgctggcgggtttccccacgttattgtctttaccatttactagttcactggagtacaatgagacaatggttttccaacaaccttctaaacaacaatcaatggtcttacaaataactgacatatacaaaacgaataatgttactttggaggacttttccaagaggcatttaaacaaagtgatttatacaagatggccatatttaagagaatccaaattggtctctttaacggatggtaagactatctatgaatatcaggagtccaatgataagaaaaagttcggattcataacgaagcctgcggaaacccaggacaaaaaacttttcaatagtttgaagaattcaatgctaaggatgtatgctaaacagaaagctgttaaaataggacctatggaagccattgctaccgtctttccagtgactggtttggtgagagactctgatggtggttatattaagacctttagccctaccccagattactatccattgcaactggttgttgaatctgttgtcaacgaggatgaaagatataaagaaagaggacccattcctattgaagaggaatttccattgaattcaaaagttattttcttaggtgattatgcctatggtggtgaaactactattgacggttacagcagtgaccgcagactaaaaattactgtagaaaagaagtttttggatagtgagcccaccatcggcaaagaaaggttacaaatggatcatcaagccgttaaatattatccgtcttatattgtgtccaagaacatgcacttacaccccttgtttttgtctaagattacttccaagttcatgattactgacgctactgggaagcatatcaatgttggtatcccggttaagttcgaagctagacaccaaaaggttttaggttacgcgaggaggaaccctaggggctgggaatactcaaatttaactctaaatttactgaaagagtatagacaaactttcccagatttttttttcaggttgtccaaggtaggtaatgatatcccagttttggaagatcttttccccgatacttccactaaggatgccatgaatttattagatggtatcaaacaatggctaaagtatgtctcatcgaagtttatcgcggtatctttggagtctgactccttaactaagacatcgattgctgccgtggaagatcatatcatgaaatacgcagctaacatcgaaggtcatgaaagaaaacagttagccaaagttcctcgtgaggctgttttgaatccaagatcatcatttgcactcttacgtagtcaaaagttcgatttgggtgaccgtgttgtttatatccaagattctggtaaggtaccaattttctcaaagggtacagttgttggctatactactctcagttcatcattatcaattcaggtcttatttgatcatgaaatcgtggccggtaataattttggcggcaggttgcgcacgaatagaggcttagggcttgatgcctctttcttattgaatattactaacaggcagttcatttatcactccaaggcttccaaaaaggctttggaaaagaaaaagcaatctaacaataggaacaataataccaaaactgctcacaagactccttcaaagcaacaatctgaagaaaaactgagaaaagaaagggcacatgatttattgaattttatcaaaaaggataccaatgaaaagaattctgaaagtgtagacaacaagagcatgggatcgcaaaaagattccaaacccgcaaagaaagttttgttaaaaagaccagctcagaaaagcagtgaaaacgtgcaagttgatttggccaattttgaaaaagcaccgcttgataatccaactgttgctggatctattttcaatgccgttgcaaatcaatattctgatggtataggcagtaatttgaatatcccaactccacctcacccaatgaatgtggttgggggtcctattcctggagcgaatgatgttgcagatgttggtttgccgtacaatattcccccaggttttatgacgcatcctaatggtcttcacccattacaccctcaccagatgccttaccctaatatgaatggaatgtctattccgccaccagcaccacatgggtttggacaaccgatttccttcccacctccacctcctatgacaaatgtttcagatcaaggaagtcgtattgttgtcaatgaaaaggaaagccaagatttgaaaaaattcattaatggtaaacagcacagcaatggttcaactattgggggagaaacaaagaacagtaggaaaggcgagattaaaccttcttctggcacaaactctactgaatgtcaatcgccaaagtcacaaagcaatgctgctgaccgtgataataaaaaagacgaatc